(19) United States
(12) Patent Application Publication
(10) Pub. No.: US 2023/0045151 A1
(43) Pub. Date: Feb. 9, 2023

(54) COMPOSITIONS AND METHODS

(71) Applicant: MONASH UNIVERSITY, Clayton, Victoria (AU)

(72) Inventors: Benjamin MARSLAND, Clayton, Victoria (AU); Tomasz Piotr WYPYCH, Clayton, Victoria (AU)

(21) Appl. No.: 17/792,042

(22) PCT Filed: Jan. 13, 2021
(86) PCT No.: PCT/AU2021/050021

§ 371 Date: Jul. 11, 2022

(30) Foreign Application Priority Data
Jan. 13, 2020 (AU) 2020900083

(51) Int. Cl. A61K 31/198; A61P 11/06; A61P 37/00; A61K 31/192; A61K 31/085

(52) U.S. Cl. CPC A61K 31/198; A61P 11/06; A61P 37/00; A61K 31/192; A61K 31/085

(57) ABSTRACT

The field of the invention relates to compositions and methods for treating and/or preventing immune dysfunction.

### FIELD OF THE INVENTION

The field of the invention relates to compositions and methods for treating and/or preventing immune dysfunction.

### BACKGROUND

Allergic asthma is a chronic airway disease characterized by the production of type 2 cytokines, synthesis of immunoglobulin E (IgE), goblet cell metaplasia, influx of inflammatory cells and ultimately, airway remodelling. Initiation of allergic asthma is a consequence of a dysregulated interplay between airway epithelium and immune cells, including dendritic cells (DCs), in response to allergen exposure. In support of this, sensing of house dust mite extract via Toll-like receptor 4 (TLR4), expressed on airway epithelial cells, has been shown to be necessary for the activation of pulmonary DCs and the initiation of allergic sensitization. The immunomodulatory properties of other receptors (and ligands) on epithelium-driven DC activation that could underpin differences in susceptibility to asthma remain obscure.

Asthma and allergic airway diseases highlight the importance of immune homeostasis. A complex system of local immune pathways maintains homeostasis within tissues such as the lungs, and there remains a need for methods and compositions to restore immune homeostasis in subjects with diseases or disorders that involve dysregulated or altered immune homeostasis.

### SUMMARY OF THE INVENTION

In one aspect the present invention provides a method of treating and/or preventing an eosinophilic disease or disorder in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In one embodiment, the present invention provides a method as described herein, wherein the eosinophilic disease or disorder in a subject is selected from the group consisting of a hypereosinophilic syndrome, eosinophilic gastritis, eosinophilic gastroenteritis, eosinophilic esophagitis, eosinophilic pneumonia, eosinophilic granulomatosis with polyangiitis, allergy, dermatitis, asthma and chronic rhinosinusitis.

In another embodiment, the present invention provides a method as described herein, wherein the eosinophilic disease or disorder in a subject is a pulmonary disease or disorder.

In a further embodiment, the present invention provides a method as described herein, wherein the eosinophilic disease or disorder in a subject is asthma.

In a further embodiment, the present invention provides a method as described herein, wherein the eosinophilic disease or disorder in a subject is allergic airway disease.

In a further embodiment, the present invention provides a method as described herein, wherein the eosinophilic disease or disorder in a subject is house dust mite associated allergic airway disease.

In a further embodiment, the present invention provides a method as described herein, wherein the administration of the therapeutically effective amount of the one or more eosinophil antagonist results in reduced eosinophilia.

In a further embodiment, the present invention provides a method as described herein, wherein the administration of the therapeutically effective amount of the one or more eosinophil antagonist results in reduced eosinophilia in bronchoalveolar lavage fluid.

In a further embodiment, the present invention provides a method as described herein, wherein the administration of the therapeutically effective amount of the one or more eosinophil antagonist results in reduced infiltration of pulmonary dendritic cells into the lungs.

In a further embodiment, the present invention provides a method as described herein, wherein the administration of the therapeutically effective amount of the one or more eosinophil antagonist results in reduced activation of pulmonary dendritic cells.

In a further embodiment, the present invention provides a method as described herein, wherein the administration of the therapeutically effective amount of the one or more eosinophil antagonist results in reduced migration of pulmonary dendritic cells into draining lymph nodes of the subject.

In a further embodiment, the present invention provides a method as described herein, wherein the administration of the therapeutically effective amount of the one or more eosinophil antagonist results in reduced goblet cell hyperplasia.

In a further embodiment, the present invention provides a method as described herein, wherein the administration of the therapeutically effective amount of the one or more eosinophil antagonist results in in reduced mucus production.

In a further embodiment, the present invention provides a method as described herein, wherein the administration of the therapeutically effective amount of the one or more eosinophil antagonist results in reduced peribronchial and/or perivascular inflammatory cell infiltrate.

In a further embodiment, the present invention provides a method as described herein, wherein the administration of the therapeutically effective amount of the one or more eosinophil antagonist results in reduced infiltration of neutrophils into the lungs.

In a further embodiment, the present invention provides a method as described herein, wherein the administration of the therapeutically effective amount of the one or more eosinophil antagonist results in reduced pathologic change in the lungs.

In a further embodiment, the present invention provides a method as described herein, wherein the administration of the therapeutically effective amount of the one or more eosinophil antagonist results in reduced production of Th2-associated cytokines.

In a further embodiment, the present invention provides a method as described herein, wherein the Th2-associated cytokines are IL-5 and/or IL-13.

In a further embodiment, the present invention provides a method as described herein, wherein the administration of the therapeutically effective amount of the one or more eosinophil antagonist results in reduced production of allergen-specific antibodies

In a further embodiment, the present invention provides a method as described herein, wherein the administration of the therapeutically effective amount of the one or more eosinophil antagonist results in reduced production of allergen-specific IgE.

In a further embodiment, the present invention provides a method as described herein, wherein the administration of the therapeutically effective amount of the one or more eosinophil antagonist results in reduced production of house dust mite specific antibodies.

In a further embodiment, the present invention provides a method as described herein, wherein the administration of the therapeutically effective amount of the one or more eosinophil antagonist results in reduced production of house dust mite specific IgE.

In a further embodiment, the present invention provides a method as described herein, wherein the administration of the therapeutically effective amount of the one or more eosinophil antagonist results in reduced T cell priming by pulmonary dendritic cells.

In a further embodiment, the present invention provides a method as described herein, wherein the administration of the therapeutically effective amount of the one or more eosinophil antagonist results in reduced CCL20 expression in airway epithelia in the subject.

In a further embodiment, the present invention provides a method as described herein, wherein the administration of the therapeutically effective amount of the one or more eosinophil antagonist results in reduced CCR6 signalling in the subject.

In a further embodiment, the present invention provides a method as described herein, wherein the administration of the therapeutically effective amount of the one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate results in reduced TLR4 signalling in the subject.

In another aspect, the present invention provides a method of reducing eosinophilia in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In a further aspect, the present invention provides a method of reducing infiltration of pulmonary dendritic cells into the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In a further aspect, the present invention provides a method of reducing activation of pulmonary dendritic cells in the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In a further aspect, the present invention provides a method of reducing migration of pulmonary dendritic cells into lymph nodes of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In a further aspect, the present invention provides a method of reducing goblet cell hyperplasia in the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In a further aspect, the present invention provides a method of reducing mucus production in the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In a further aspect, the present invention provides a method of reducing a peribronchial and/or perivascular inflammatory cell infiltrate in the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In a further aspect, the present invention provides a method of reducing infiltration of neutrophils into the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In a further aspect, the present invention provides a method of reducing pathologic change in the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In a further aspect, the present invention provides a method of reducing Th2-associated cytokine production in the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate. In one embodiment the Th2-associated cytokine is IL-5 and/or IL-13

In a further aspect, the present invention provides a method of reducing the production of allergen specific antibodies in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In a further aspect, the present invention provides a method of reducing the production of house dust mite specific antibodies in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In one embodiment the antibodies are IgE antibodies.

In a further aspect, the present invention provides a method of reducing the priming of T cells by pulmonary dendritic cells in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In a further aspect, the present invention provides a method of reducing CCL20 expression in airway epithelia in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In a further aspect, the present invention provides a method of reducing CCR6 signalling in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In a further aspect, the present invention provides a method of reducing TLR4 signalling in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In one embodiment EGFR-TLR4 cross talk is reduced.

In a further aspect, the present invention provides a method of reducing EGFR mediated signalling in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In a further aspect, the present invention provides a method of reducing LPS-induced septic shock in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In a further embodiment, the present invention provides a method as described herein wherein L-tyrosine, N-acetyl-L-tyrosine, L-DOPA, 4-hydroxyphenylpyruvate, 4-hydroxyphenylacrylate, 3-(p-hydroxyphenyl)propionate, p-cresol and/or p-cresol sulphate is produced in the subject following administration of the one or more eosinophil antagonist.

In a further embodiment, the present invention provides a method as described herein, wherein the therapeutically effective amount of the one or more eosinophil antagonist is administered in two or more doses.

In a further embodiment, the present invention provides a method as described herein, wherein the therapeutically effective amount of the one or more eosinophil antagonist is administered daily, weekly, biweekly, bimonthly, and or quarterly.

In a further embodiment, the present invention provides a method as described herein, wherein the subject is administered with a therapeutically effective amount of the one or more eosinophil antagonist is treated before, during, after, or simultaneously with one or more additional therapies for the treatment of the eosinophilic disease or disorder.

In a further embodiment, the present invention provides a method as described herein, wherein the therapeutically effective amount of the one or more eosinophil antagonist is administered orally, by inhalation, intravenously, intramuscularly, subcutaneously, topically or a combination thereof.

In a further embodiment, the present invention provides a method as described herein, wherein the one or more eosinophil antagonist is formulated as a composition further comprising one or more pharmaceutically acceptable excipients.

In a further aspect, the present invention provides a composition comprising one or more eosinophil antagonists for use in the treatment and/or prevention of a pulmonary disease in a subject, wherein the one or more eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In a further embodiment, the present invention provides a method as described herein, or a composition as described herein, wherein the composition consists of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In a further aspect, the present invention provides a use of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate in the manufacture of a medicament for treating an eosinophilic disease or disorder in a subject.

### BRIEF DESCRIPTION OF DRAWINGS

FIG. 1 shows mice with restricted antibody repertoire to hen egg lysozyme (MD4) fail to mount allergic responses to house dust mite. a, Differential cell counts in the BALF. Mac, macrophages; neutr, neutrophils; eos, eosinophils; lymph, lymphocytes. b, Total number of dendritic cells in the lungs and their surface expression of PD-L2. GMFI, geometric mean fluorescence intensity. c, Representative Periodic acid-Schiff (PAS)-stained lung tissue from WT or MD4 mice and the quantification of the frequency of PAS+ bronchi in histological sections. Scale bars, 100 μM, p=0.0074. d, Representative H&E-stained lung tissue from WT or MD4 mice. Scale bars, 100 μM. e, Concentration of IL-5 and IL-13 in culture supernatants of mediastinal lymph node cells re-stimulated with the indicated concentrations of HDM for 4 days, ***p=0.0005 (IL-5), **p=0.0012 (IL-13). f, Total number of CD4+ T cells and the frequency of Treg cells (expressed as the percentage of CD4+ T cells) in the lung tissue, **p=0.0063 (CD4+322 T cells), *p=0.0495 (Treg cells). g, Principal coordinate analysis (PCoA) plot (based on Bray-Curtis distance) of the bacterial communities (as determined by sequence analysis of 16S rRNA gene amplicons) in WT and MD4 fecal samples. All data except in d, g are expressed as the mean±s.e.m (error bars shorter than the size of the symbols in e are not depicted). Data in a, f are pooled from 4 experiments (n=19 biologically independent samples per group), data in b are pooled from 3 experiments (n=14 biologically independent samples per group) data in c, d are representative of 2 experiments (n=8 biologically independent samples per group), data in e are pooled from 2 experiments (n=9 biologically independent samples per group), data in g are pooled from 5 experiments (n=37 MD4, n=29 WT biologically independent samples). Statistical significance for a-c, f was evaluated with two-sided unpaired Student's t-test (in the case of Gaussian distribution) or Mann-Whitney test (non-Gaussian distribution). Statistical significance for e was determined with Two-Way analysis of variance (ANOVA) with Sidak correction for multiple comparisons. Data distribution was assessed with D'Agostino & Pearson normality test. Statistical significance for g was evaluated with an Analysis of Similarities (ANOSIM) controlling for experimental variation. *p≤0.05, **p≤0.01, ***p≤0.001, ****p≤0.0001).

FIG. 2 shows microbiota of the MD4 mice confers protection against HDM-induced allergic airway inflammation. a, PCoA plot (Bray-Curtis distance) of the bacterial communities (16S rRNA gene amplicons) in mouse faeces. b, Differential cell counts in the BALF, p=0.0306. c, Representative H&E-stained lung tissue from GF-WT or GF-MD4 mice. Scale bars, 100 μM. d, Representative PAS-stained lung tissue from GF-WT or GF-MD4 mice and the quantification of the frequency of PAS+ bronchi in histological sections, *p=0.0102. Scale bars, 100 μM. e, Cytokine concentration in culture supernatants of mediastinal lymph node cells re-stimulated with the indicated concentrations of HDM for 4 days, ***p=0.0004 (IL-13). f, Levels of HDM-specific IgG1 antibodies in the serum, *p=0.0235. g, Total number of CD4+ T cells and the frequency of Treg cells (percentage of CD4+ T cells) in the lungs. Results are pooled from two experiments (n=10 biologically independent samples per group), except from data in d and f, where data is representative of two experiments (n=5 biologically independent samples per group). All data except in a, c are presented as mean values±s.e.m (error bars shorter than the size of the symbols in e are not depicted). Statistical analysis was performed as per FIG. 1. *p≤0.05, ***p≤0.001, ****p≤0.0001.

FIG. 3 shows antibody cross-reactivity shapes the microbiome and the metabolome of the host. a, A heat map representing differentially abundant ASVs between MD4 and WT mice using Zero-inflated Gaussian mixture model controlling for experimental variation. In italics, MD4 IgA-bound hits analyzed in b. b, Correlation inference network with bacterial taxa bound by anti-HEL IgA (annotated). Blue or black nodes represent taxa differentially abundant in the MD4 or WT mice, respectively, while open nodes represent non-differentially abundant hits. Node size is proportional to the IgA binding index calculated from IgA+ and IgA− fractions. c, Volcano plot depicting the differential abundance of plasma metabolites between WT and MD4 mice using limma parametric empirical Bayes (eBayes) testing. Y axis represents the −log 10 adjusted p-value (with dashed line at α=0.05) while X axis represents the log 2 fold change (dashed line at 2-fold change). d, pathway of L-tyrosine conversion to PCS by ThiH. e, levels of PCS in WT, MD4 and GF mice co-housed with WT or MD4 mice, *p=0.0235 (WT vs MD4), *p=0.0155 (GF-WT vs GF-MD4). f, volcano plot representing differences in bacterial genes abundance between WT and MD4 mice. g, levels of L-tyrosine in the faeces of WT and MD4 mice, **p=0.0041. Data in a are pooled from 5 experiments (n=37 MD4, n=27 WT biologically independent samples), IgA binding data in b represent analysis from three independent sorting experiments (n=3 biologically independent samples per group), data in c are from one experiment (n=8 biologically independent samples per group), data in e are representative of 2 two experiments (n=5 biologically independent samples per group), data in f represent samples with the highest quality DNA from 4 pooled experiments (n=11 MD4, n=9 WT biologically independent samples), while data in g are pooled from two independent experiments (n=9 MD4, n=11 WT biologically independent samples). Data in e, g are presented as mean values±s.e.m. Statistical analysis was performed as per FIG. 1. *p≤0.05, **p≤0.01.

FIG. 4 shows administration of PCS or L-tyrosine confers protection in an HDM model of asthma. a, Differential cell counts in the BALF of vehicle or PCS-treated mice (as indicated in the Methods section), **p=0.0052. b, Total number of DCs in the lungs, ***p=0.0007. c, Cytokine concentration in culture supernatants of mediastinal lymph node cells re-stimulated with HDM for 4 days, ***p=0.0004 (IL-5), **p=0.0025 (IL-13). d, Total number of CD4+ T cells and the frequency of Treg cells (percentage of CD4+ T cells) in the lungs. e, Differential cell counts in the BALF of vehicle or L-tyrosine-treated mice (as indicated in the Methods section), *p=0.018 (Neutr), **p=0.0019 (Eos). f, Total number of DCs in the lungs, ***p=0.0008. g, Cytokine concentration in culture supernatants of mediastinal lymph node cells re-stimulated with HDM for 4 days, *p=0.0185 (IL-13). h, Total number of CD4+ T cells and i, the frequency of Treg cells (percentage of CD4+ T cells) in the lungs. Results in a, b are pooled from 4 experiments (n=18 biologically independent samples per group), results from c are pooled from 3 experiments (n=14 biologically independent samples per group), data from d-h are pooled from two experiments (n=9 biologically independent samples per group), while data in i are representative of one experiment (n=5 biologically independent samples per group). All data are presented as mean values±s.e.m. Statistical analysis was performed as per FIG. 1. *p≤0.05, **p≤0.01, ***p≤0.001.

FIG. 5 shows the L-tyrosine—PCS axis modulates DC activation via inhibition of epithelial cell derived CCL20. a, Surface expression of CD80, CD86 and PD-L2 on HDM-positive and HDM-negative population of lung DCs. b, HDM uptake in vivo by lung DCs from vehicle or L-tyrosine treated mice, *p=0.0367. c, Migration of lung DCs to lung draining lymph nodes, **p=0.0014. d, capacity of pulmonary DCs from L-tyrosine or vehicle-treated groups to prime OT-II cells from a naïve mouse into an IL-13-producing subset (left) or restimulate effector Th cells from HDM-treated mice as per FIG. 1 (right). e, capacity of PCS to modulate HDM-induced secretion of chemokines from lung cells isolated from naïve mice. f, capacity of PCS to inhibit CCL20 secretion from LPS-stimulated lung cells from naïve mice, p=0.0079. g, Concentration of CCL20 in the BALF of mice treated as per FIG. 4e, *p=0.032. h, CCL20 production by lung cells stimulated with LPS in the presence of PCS (***p=0.0005), EGF (*p=0.043), AREG (*p=0.0278) or Gefitinib (**p=0.0053). Data from a-c are pooled from two experiments (a, b, n=10 biologically independent samples per group; c, n=9 biologically independent samples per group). Data in d represent technical replicates (n=6 left panel, n=5 right panel) from one experiment. Data in e are pooled from 3 independent experiments (n=6 per group). Data in f are pooled from two independent experiments (n=5 biologically independent samples per group). Data in g are pooled from two independent experiments (n=9 biologically independent samples per group). Data in h are pooled from two independent experiments (n=4 biologically independent samples per group). All data are presented as mean values±s.e.m. Statistical significance for a-d, f, g, was evaluated with unpaired Student's t-test (in the case of Gaussian distribution) or Mann-Whitney test (non-Gaussian distribution). Statistical significance for e, h was determined with One-Way analysis of variance (ANOVA) with Dunnett correction for multiple comparisons. Data distribution was assessed with D'Agostino & Pearson normality test. *p≤0.05, **p≤0.01, ***p≤0.001, ****p≤0.0001

FIG. 6 shows T helper cells from the MD4 mice do not acquire Th2 phenotype upon intranasal exposure to HDM. a, Cytokine concentrations in culture supernatants from co-cultures of DCs and in vivo-primed lung CD4+CD44+ T cells restimulated with HDM for 4 days. b, Total numbers of eosinophils, dendritic cells and surface expression of PD-L2 on dendritic cells from WT or B cell-deficient (JhT) mice exposed to HDM as per FIG. 1. Data in a, are representative of two experiments and represent technical replicates (n=4 WT, n=4 MD4). Data in b are pooled from 2 experiments (n=8 per group), or are representative of 2 experiments (PD-L2 expression) (n=4 per group). All data are presented as mean values+/−SEM.

FIG. 7 shows MD4 mice harbour diverse microbiota. Alpha diversity measure (based on Shannon and Chao1 indexes) based on 16S rDNA amplicons in WT and MD4 faecal samples. Data pooled from 5 experiments (n=37 MD4, n=27 WT).

FIG. 8 shows levels and specificity of secretory antibodies in the faeces of MD4 mice. Quantification of antibody levels in the faeces of WT or MD4 mice and their reactivity to HEL. All data are pooled from two experiments, n=11 WT, n=13 MD4. All data are presented as mean values+/−SEM. Statistical significance was evaluated with unpaired Student's t-test (in the case of Gaussian distribution) or Mann-Whitney test (non-Gaussian distribution). Data distribution was assessed with D'Agostino & Pearson normality test. ****p≤0.0001.

FIG. 9 shows the correlation inference network with annotated bacterial taxa bound by anti-HEL IgM (blue font) within MD4 microbiota. Blue nodes represent taxa differentially abundant in the MD4 or WT mice, respectively, while open nodes represent non-differentially abundant hits. Node size is proportional to the MD4 IgM binding index calculated from IgM+ and IgM− fractions. Data represent analysis from one sorting experiment.

FIG. 10 shows taxonomic analyses of WT and MD4 bacteria using shotgun metagenomics. A heat map representing differentially abundant species between MD4 and WT mice. Data represent samples with the highest quality DNA from 4 pooled experiments (n=11 MD4, n=9 WT).

FIG. 11 shows shotgun metagenomics analyses of metabolic pathways from tyrosine to p-cresol. a, Metabolic pathways related to tyrosine conversion to p-cresol by bacteria. Enzymes: tyrosine lyase (ThiH), tyrosine aminotransferase B (TyrB), phenyllactate dehydrogenase (FldH), phenyllactate dehydratase (FldBC), acyl-CoA dehydrogenase (AcdA), pyruvate ferredoxin oxidoreductase A (PorA) and hydroxyphenylacetate decarboxylase (Hpd). Unknown enzymes are indicated by a question mark. b, Volcano plot depicting differential abundance of bacterial genes related to p-cresol production from tyrosine in fecal samples from WT and MD4 mice. Each color (squares in a and dots in b) represents a different gene encoding for an enzyme or enzyme subunit of the described pathways. TyrB, PorA, and FldH were not found in metagenomics data. Data represent samples with the highest quality DNA from 4 pooled experiments (n=11 MD4, n=9 WT).

FIG. 12 shows PCS concentration increases in the faeces and in the airways of L-tyrosine-fed mice. Mice were fed with L-tyrosine in drinking water (100 mg/kg/day) for 14 days, after which faeces were collected. BALF samples were collected after HDM immunization as per FIG. 1. PCS was measured using LC-MS targeted metabolomics (n=5 per group). Data represent samples from one experiment. All data are presented as mean values+/−SEM.

FIG. 13 shows microbiota depletion abrogates the beneficial 423 effect of L-Tyrosine feeding. a, Experimental setup: WT C57BL6/J mice were treated with a combination of enrofloxacin (Baytril®) and amoxicillin with clavulanic acid for one week and maintained on amoxicillin/clavulanic acid until end of experiment. L-tyrosine treatment was initiated 2 weeks after the antibiotic treatment until end of experiment b, total number of eosinophils in the BALF and lungs, p=0.0342 (BALF), p=0.0173 (Lungs), c, total number of DCs in the lungs d, concentrations of IL-5 in the BALF, p=0.042; n=5 per group for all except for Water/Water group in b and d where n=4. Results are representative of two independent experiments. All data are presented as mean values+/−SEM. Statistical significance was evaluated with unpaired Student's t-test (in the case of Gaussian distribution) or Mann-Whitney test (non-Gaussian distribution) Data distribution was assessed with Kolmogorov-Smimov normality test.

FIG. 14 shows MD4 mice have impaired production of CCL20 upon HDM exposure. a, CCL20 levels in culture supernatants of lung cells isolated from WT or MD4 mice and stimulated in vitro with HDM, p=0.0002. b, CCL20 concentration in BALF of WT or MD4 mice 2 hours after intranasal exposure to HDM, p=0.032. N=6 per group for all graphs except from MD4 group in b, where n=5. Results are pooled from two independent experiments. All data are presented as mean values+/−SEM. Statistical significance was evaluated with unpaired Student's t-test (in the case of Gaussian distribution) or Mann-Whitney test (non-Gaussian distribution). Data distribution was assessed with Kolmogorov-Smirnov normality test. *p≤0.05, ***p≤0.001.

FIG. 15 shows administration of PCS confers protection in an OVA/LPS model of pulmonary type 1 response. a, Experimental setup of PCS administration in a protocol of OVA/LPS exposure. b, Numbers of neutrophils, CD4+ and CD8+438 T cells in the BALF of vehicle or PCS-treated mice. Results are from one experiment (n=4 per group, p=0.0335). All data are presented as mean values+/−SEM. *p≤0.05. Statistical significance was evaluated with Mann-Whitney test.

### DETAILED DESCRIPTION

Eosinophils are a key effector cell in the pathology of eosinophilic diseases and disorders.

The present invention is based in part on the discovery that eosinophilic disease can be treated and/or prevented by administering to a subject L-tyrosine and/or p-cresol sulphate (PCS).

For example, Example 3 demonstrates that transfer of the PCS-producing microbiota ameliorated eosinophilia, production of HDM-specific antibodies, lung pathology, goblet cell hyperplasia, mucus production, and secretion of Th2-associated cytokines. Example 5 demonstrates that administration of PCS or L-tyrosine protects against allergic airway inflammation, in particular, reduced eosinophilia in the BALF, decreased infiltration of DCs into the lungs, and reduced production of IL-5 and IL-13 by restimulated mediastinal lymph nodes. Example 6 demonstrates that administration of tyrosine reduces activation of pulmonary dendritic cells and reduces migration of dendritic cells to the draining lymph nodes.

Accordingly, in one aspect the present invention provides a method of treating and/or preventing an eosinophilic disease or disorder in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

Phenols (phenol and p-cresol) are microbial metabolites produced from tyrosine metabolism. A non-essential amino acid, in animals, L-tyrosine is synthesized from phenylalanine. L-phenylalanine is an essential amino acid.

The present inventors have demonstrated herein that the tyrosine metabolism related molecules tyrosine and p-cresol sulphate have activity in vivo, and the present inventors propose that the intermediates in the metabolic pathways from tyrosine to p-cresol, products of p-cresol metabolism (p-cresol glucuronide and p-cresol sulfate) as well as phenylalanine which is upstream of tyrosine, can be used in the methods described herein.

Phenol exhibits cytotoxicity and increases paracellular permeability in vitro; it acts as a promoter of skin cancer in an animal model. Previously, p-cresol has been shown to exhibit cytotoxicity and genotoxicity and reduces endothelial barrier function in vitro. Increases in levels of p-cresol sulfate (PCS; a sulfate-conjugate of p-cresol) a microbial metabolite derived from secondary metabolism of p-cresol, is found in urine.

PCS is associated with chronic kidney disease-associated events such as cardiovascular disease and appears to be elevated in the urine of individuals with progressive multiple sclerosis. Furthermore, phenol and p-cresol have previously been implicated in suppressing the differentiation of keratinocytes in humans and causing dermal disorders in mice. Surprisingly, the present inventors demonstrate herein that a metabolite of p-cresol, PCS, is not deleterious to epithelial cells, dendritic cells, macrophages and bone marrow precursors.

4-hydroxyphenylpyruvate (4-HPPA) is a keto acid that is involved in the tyrosine catabolism pathway. It is a product of the enzyme (R)-4-hydroxyphenyllactate dehydrogenase (EC1.1.1.222) and is formed during tyrosine metabolism.

4-hydroxyphenylacrylate is formed from 4-hydroxyphenylpyruvate by the action of the intestinal microbial enzyme FldH.

3-(p-hydroxyphenyl)propionate is another product of tyrosine metabolism, and is formed from 4-hydroxyphenylacrylate by the action of the intestinal microbiota enzymes FldBC and AcdA. Notably, 3-(p-hydroxyphenyl)propionate is an irritant, and may cause respiratory tract irritation.

As discussed above, surprisingly, the present inventors demonstrate herein that a metabolite of p-cresol, PCS, is not deleterious to epithelial cells, dendritic cells, macrophages and bone marrow precursors. P-cresol glucuronide (PCG) is a second product of p-cresol metabolism (next to p-cresol sulfate). It is produced in reduced concentration than p-cresol sulfate.

Ni et al. (2014) Therapeutic Apheresis and Dialysis, 18(6):637-642 describes that uremic toxins such as p-cresol sulfate (PCS) are associated with increased mortality for chronic kidney disease (CKD) patients. In particular, free PCS was reported to be associated with an increased risk of general and cardiovascular-related mortality in CKD patients. Furthermore, PCS toxicity has been established in vitro, with PCS being deleterious to leukocytes, endothelial cells, and renal tubular cells.

Kelly et al. (2018) Clin Exp Allergy. 2018; 48:1297-1304 demonstrated there was no evidence of a systematic difference in the metabolome of children reporting current asthma vs. healthy controls according to partial least squares discriminant analysis. However, p-cresol sulphate was associated with decreased odds of current asthma at a nominally significant threshold. Kelly et al. indicates that p-cresol sulphate may be an indicator of a gut microbiome enterotype, and does not determine a connection between the gut microbiome, the circulating metabolome and their relationship to asthma. For example, the data presented herein demonstrate immunological changes can cause an alteration in the gut microbiome, leading to a change in the circulating metabolome, causing an effect in levels of PCS.

Wyczalkowska-Tomasik et al. (2016) Geriatr Gerontol Int. 17:1022-1026 demonstrates that age-dependent increase in serum levels of the toxin p-cresol sulphate is not related to their precursor tyrosine. In contrast, the data presented herein demonstrates that administration of the PCS precursor tyrosine results in the same effects as the administration of PCS.

Lee-Sarwar et al. (2019) J ALLERGY OLIN IMMUNOL 144(2): 442-453 describes metabolites, including p-cresol sulfate, that have an association with reduced risk of asthma, and also demonstrated that exclusive breastfeeding for the first 4 months inversely correlated with asthma, and PCS, which positively correlated with breastfeeding, explained 17.3% of this effect (Table E4 of Lee-Sarwar et al.).

Although PCS is reported as a uremic toxin in chronic kidney disease (CKD) patients, it is also present in the blood of healthy people. In mice with normal renal function, intraperitoneally or orally administered PCS is cleared from the blood within 4 hours, and chronic administration of PCS (twice a day for 4 weeks) does not lead to its accumulation. Hence, the present inventors propose that PCS has detrimental effects only when kidney function is impaired or that it is primarily a biomarker of this condition. Of note, an elevation of any major kidney toxicity markers upon PCS treatment was not detected (Data not shown).

In various diseases or disorders, eosinophils are increased in the peripheral blood and/or tissues, a condition referred to as eosinophilia.

As used herein, the term “eosinophilic disease or disorder” includes any disease or disorder characterized by an elevated level of eosinophils in blood, a tissue, or an organ, such as the lungs. Methods for determining eosinophil levels, such as normal and abnormal (e.g., elevated) eosinophil levels in the eosinophilic diseases or disorders disclosed herein are known in the art.

Examples of eosinophilic diseases and disorders include a pulmonary disease or disorder, asthma, allergic airway disease, house dust mite associated allergic airway disease, hypereosinophilic syndrome, eosinophilic gastritis, eosinophilic gastroenteritis, eosinophilic esophagitis, eosinophilic pneumonia, eosinophilic granulomatosis with polyangiitis, allergy, dermatitis, asthma and chronic rhinosinusitis.

Accordingly, in one embodiment, the eosinophilic disease or disorder in a subject is selected from the group consisting of a hypereosinophilic syndrome, eosinophilic gastritis, eosinophilic gastroenteritis, eosinophilic esophagitis, eosinophilic pneumonia, eosinophilic granulomatosis with polyangiitis, allergy, dermatitis, asthma and chronic rhinosinusitis.

In another embodiment, the eosinophilic disease or disorder in a subject is a pulmonary disease or disorder.

As used herein, the term “pulmonary disease or disorder” refers to a disease or disorder with pathology affecting at least in part the lungs or respiratory system characterized by an elevated level of eosinophils.

Examples of pulmonary diseases or disorders include asthma, allergic airway disease, house dust mite associated allergic airway disease allergic rhinitis, chronic rhinosinusitis.

In one embodiment, the eosinophilic disease or disorder in a subject is asthma.

As used herein, the term “asthma” refers to diseases or disorders that present as reversible airflow obstruction and/or bronchial hyper-responsiveness that may or may not be associated with underlying inflammation.

Examples of asthma include allergic asthma, atopic asthma, corticosteroid naive asthma, chronic asthma, corticosteroid resistant asthma, corticosteroid refractory asthma, asthma due to smoking, asthma uncontrolled on corticosteroids and other asthmas.

In one embodiment, the eosinophilic disease or disorder in a subject is allergic airway disease.

Allergic airway diseases include allergic rhinitis, chronic rhinosinusitis, and asthma, and show high prevalence in children.

House dust mites (HDM; Dermatophagoides sp.) are one of the commonest aeroallergens worldwide and up to 85% of asthmatics are typically HDM allergic. Allergenicity is associated both with the mites themselves and with ligands derived from mite-associated bacterial and fungal products.

In one embodiment the eosinophilic disease or disorder in a subject is house dust mite associated allergic airway disease.

Other aeroallergens include grass, weed and tree pollens, fungal spores, animal allergens (e.g. animal dander).

In another embodiment, the eosinophilic disease or disorder in a subject is allergic airway disease associated with one or more aeroallergen selected from the group consisting of a grass pollen, a weed pollen, a tree pollen, a fungal spore, and an animal allergen.

In one embodiment, the subject is an individual who has, or has had at any time in the past, clinical symptoms of allergic airway disease, such as house dust mite associated allergic airway disease, and/or sensitization to an allergen and/or an allergen-specific IgE response, or an individual at risk of developing such symptoms. Sensitisation to an allergen may be assessed by detecting IgE directed against allergen(s) from this source in the serum of the patient or by skin testing with a preparation containing the corresponding allergen(s). The allergens include a house dust mite allergen and ligands derived from mite associated bacterial and fungal products.

As used herein, the term “treating” includes reducing the level of eosinophils in blood, a tissue, or an organ, such as the lung of the subject, reducing the occurrence of the eosinophilic disease or disorder in the subject, and/or reducing the severity of the eosinophilic disease or disorder in the subject. Treating also includes decreasing at least one clinical symptom of the eosinophilic disease or disorder in the subject. Similarly, for other diseases or disorders, the term “treating” includes improving at least one symptom and/or measure of the disease or disorder.

As used herein, the term “preventing” includes preventing an elevated level of eosinophils in blood, a tissue, or an organ of the subject, such as the lung, from occurring, preventing the occurrence of the eosinophilic disease or disorder in the subject, and/or preventing an episode of the eosinophilic disease or disorder in the subject. Preventing also includes delaying the onset of at least one clinical symptom, preventing the worsening of at least one clinical symptom and/or delaying the progression of at least one clinical symptom of the eosinophilic disease or disorder in the subject. Similarly, for other diseases or disorders, the term “preventing” includes preventing or delaying at least one symptom and/or measure of the disease or disorder.

For example, in the case of asthma, a clinical symptom or measure includes an asthma exacerbation in the subject.

As used herein, the term “subject” refers to refers to a human or nonhuman animal that would benefit from the treatment and/or prevention of an eosinophilic disease or disorder or a clinical symptom of the eosinophilic disease or disorder. The term includes subjects with an eosinophilic disease or disorder or a clinical symptom of the eosinophilic disease or disorder and/or subjects at risk of developing an eosinophilic disease or disorder or a clinical symptom of the eosinophilic disease or disorder.

In one embodiment. the subject has a high level of eosinophils. For example, the patient has a level of blood eosinophils of >150 cells/L.

In another embodiment. the subject has a low level of eosinophils. For example, the patient has a level of blood eosinophils of <150 cells/L.

As used herein, the “eosinophil antagonist” refers to a compound which can directly or indirectly:
(i) inhibit, lessen, or prevent an activity of eosinophils in the subject;
(ii) inhibit, reduce, or deplete eosinophil numbers/levels (e.g. eosinophilia), including in the subject, either systemically or in a specific tissue or organ (such as the lung);
(iii) reduce the half-life of eosinophils in the subject; and/or
(iv) prevent exacerbation of symptoms associated with elevated levels of eosinophils or an activity of eosinophils in the subject.

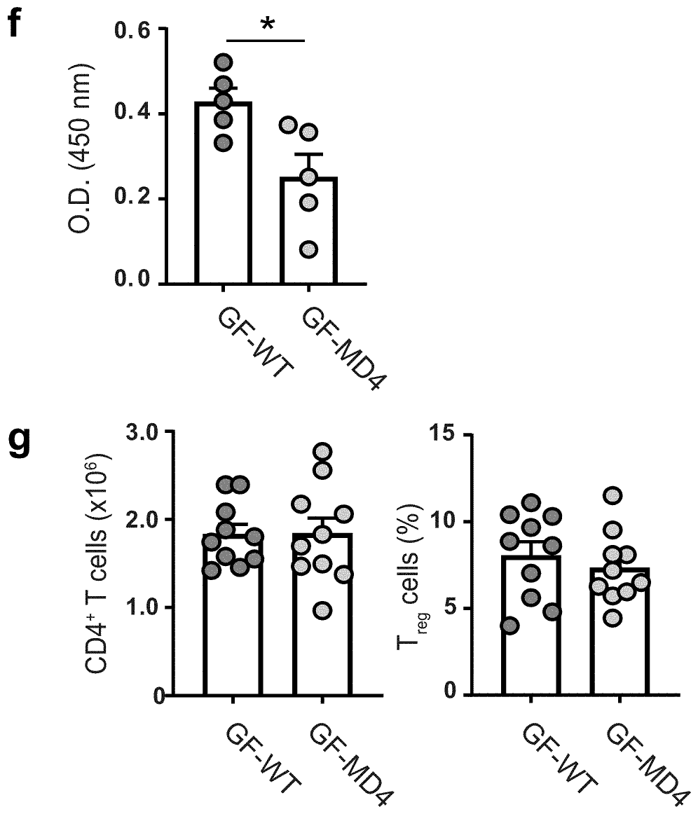

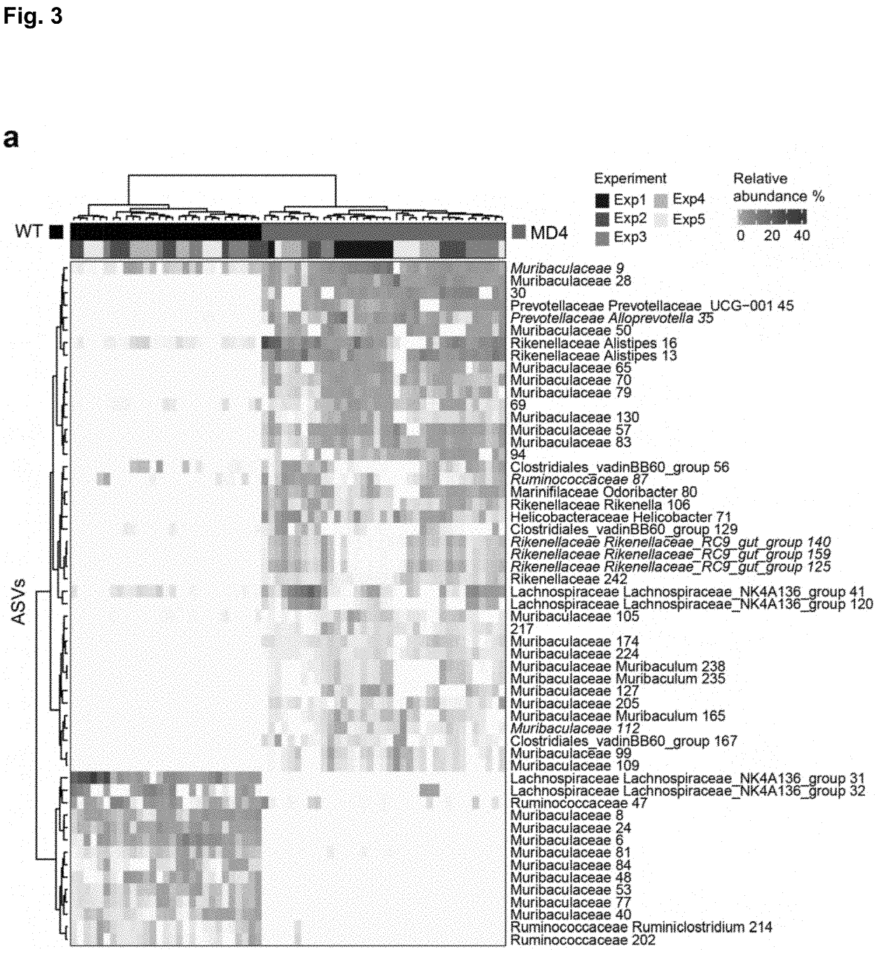

As is shown in FIG. 3, PCS is a microbial-derived end product of L-tyrosine metabolism, whereby PCS is produced from L-tyrosine via 4-hydroxyphenylpyruvate, 4-hydroxyphenylacrylate, and 3-(p-hydroxyphenyl)propionate. L-tyrosine can be produced from L-phenylalanine metabolism.

Acetyltyrosine (N-acetyl-L-tyrosine) converts to tyrosine. Accordingly, in one embodiment, the eosinophil antagonist is N-acetyl-L-tyrosine.

The eosinophil antagonists described herein can be administered as other forms that can be converted into to the eosinophil antagonist.

In another embodiment, the eosinophil antagonist is structurally similar to an eosinophil antagonist described herein. For example, L-tyrosine is converted to levodopa (L-DOPA) by the enzyme tyrosine hydroxylase; L-DOPA is structurally similar to L-tyrosine, lacking one hydroxyl group relative to L-tyrosine. Accordingly, in one embodiment, the eosinophil antagonist is L-DOPA.

Accordingly, in one embodiment, the present invention provides methods as described herein wherein the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and/or p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

As used herein the term “therapeutically effective amount” refers to an amount of the one or more eosinophil antagonist that is effective to produce a desired effect, such as providing a prevention, delay, reduction or mitigation of at least one clinical symptom of a disease or disorder in a subject. For example, an eosinophilic disease or disorder in a subject.

For example, when the disease or disorder is asthma, a therapeutically effective amount is the quantity which, when administered, produces a desired effect, such as improves the prognosis and/or state of the subject and/or that reduces or inhibits one or more symptoms of asthma to a level that is below that observed and accepted as clinically diagnostic or clinically characteristic of that condition. Alternatively, a therapeutically effective amount is a quantity which, when administered, prevents the occurrence or exacerbation of one or more symptoms of asthma. The amount to be administered will depend on the particular characteristics of the subtype of asthma to be treated, the type and stage of condition being treated, the mode of administration, and the characteristics of the subject, such as general health, other diseases, age, sex, genotype, and body weight. A person skilled in the art will be able to determine appropriate dosages depending on these and other factors.

In one embodiment the desired effect is inhibition, lessening, or prevention of an activity of eosinophils in the subject. In another embodiment, the desired effect is inhibition, reduction, or the depletion of eosinophil numbers/levels (e.g. eosinophilia), including in the subject, either systemically or in a specific tissue or organ (such as the lung). In another embodiment, the desired effect is a reduction in the half-life of eosinophils in the subject. In another embodiment, the desired effect is prevention of exacerbation of symptoms associated with elevated levels of eosinophils or an activity of eosinophils in the subject.

The present inventors have demonstrated in Example 5 that oral administration of L-tyrosine reduces eosinophilia and intravenous administration of PCS ameliorated the eosinophilia in the BALF.

Accordingly, in one embodiment, the present invention provides a method as described herein wherein the administration of the therapeutically effective amount of the one or more eosinophil antagonist results in reduced eosinophilia.

As used herein, the term “reduced” refers to a level or range that is lower than the level or range prior to administration of the therapeutically effective amount of the one or more eosinophil antagonists, or lower than the level or range in a control, or a specified threshold.

As used herein, the term “increased” refers to a level or range that is higher than the level or range prior to administration of the therapeutically effective amount of the one or more eosinophil antagonists, or lower than the level or range in a control, or a specified threshold.

A normal level or range, or a specified threshold, can be defined in accordance with standard practice.

In one embodiment, the relevant control is a sample obtained from an individual with no detectable symptoms of an eosinophilic disease or disorder.

Example 3 demonstrates that transfer of PCS-producing microbiota ameliorated eosinophilia, production of HDM-specific antibodies, lung pathology, goblet cell hyperplasia, mucus production, and secretion of Th2-associated cytokines. Example 5 demonstrates that administration of PCS prior to house dust mite sensitisation and challenge ameliorated eosinophilia in bronchoalveolar lavage fluid. Accordingly, in one embodiment the present invention provides a method as described herein, wherein the administration of the therapeutically effective amount of the one or more eosinophil antagonist results in reduced eosinophilia in bronchoalveolar lavage fluid.

In another embodiment, the therapeutically effective amount of the one or more eosinophil antagonist results in reduced eosinophilia in bronchoalveolar lavage fluid in an airway, the lungs, the trachea, or the blood. Eosinophilia may also be reduced in a body part affected by an allergy, such as eyes, skin, and gut.

In one embodiment, the level or range of eosinophilia following administration of one or more eosinophil antagonists is at least about 5%, at least about 10%, at least about 15%, at least about 20%, at least about 25%, at least about 30%, at least about 35%, at least about 40%, at least about 45%, at least about 50%, at least about 55%, or at least 60%, at least about 65%, at least about 70%, at least about 75%, at least about 80%, at least about 85%, at least about 90%, at least about 95% compared to the level or range prior to administration of the therapeutically effective amount of the one or more eosinophil antagonists, or lower than the level or range in a control, for example, the level or range in a population of patients treated with a placebo, or lower than a specified threshold.

Initiation of allergic asthma is a consequence of a dysregulated interplay between airway epithelium and immune cells, including dendritic cells (DCs), in response to allergen exposure.

Example 5 demonstrates that intravenous injection of PCS prior to HDM sensitisation decreased infiltration of neutrophils and dendritic cells into the lungs, and oral administration of L-tyrosine reduced DC recruitment. Example 6 demonstrates that administration of tyrosine reduces activation of pulmonary dendritic cells, and the reduces migration of dendritic cells to the draining lymph nodes. In particular, Example 6 demonstrates that following administration of L-tyrosine reduced the capacity of dendritic cells to prime naïve CD4+ T cells or restimulate in vivo-primed effector T helper cells into an IL-13-producing subset.

Accordingly, in one embodiment the present invention provides a method as described herein wherein the administration of the therapeutically effective amount of the one or more eosinophil antagonist results in reduced infiltration of pulmonary dendritic cells into the lungs.

In another embodiment the present invention provides a method as described herein wherein the administration of the therapeutically effective amount of the one or more eosinophil antagonist results in reduced activation of pulmonary dendritic cells.

In one embodiment, pulmonary dendritic cell activation is measured by measuring the ability of pulmonary dendritic cells to prime CD4+ T cells.

In another embodiment, pulmonary dendritic cell activation is measured by measuring the ability of pulmonary dendritic cells to increase IL-13 levels.

Accordingly, in one embodiment, the present invention provides a method as described herein wherein the administration of the therapeutically effective amount of the one or more eosinophil antagonist results in reduced T cell priming by pulmonary dendritic cells.

As is shown in Example 6, the migratory capacity of DCs was decreased, as shown by a reduced frequency of HDM+DCs in the draining LNs. Accordingly, in another embodiment, the present invention provides a method as described herein wherein the administration of the therapeutically effective amount of the one or more eosinophil antagonist results in reduced migration of pulmonary dendritic cells into draining lymph nodes of the subject.

As used herein, the term “dendritic cell migration” includes the level of migration of dendritic cells from one location to another in vivo.

In one embodiment, the level or range of dendritic cell activity (e.g. infiltration into the lungs, activation, migration into the draining lymph nodes) following administration of one or more eosinophil antagonists is at least about 5%, at least about 10%, at least about 15%, at least about 20%, at least about 25%, at least about 30%, at least about 35%, at least about 40%, at least about 45%, at least about 50%, at least about 55%, or at least 60%, at least about 65%, at least about 70%, at least about 75%, at least about 80%, at least about 85%, at least about 90%, at least about 95% compared to the level or range of dendritic cell activity (e.g. infiltration into the lungs, activation, migration into the draining lymph nodes) prior to administration of the therapeutically effective amount of the one or more eosinophil antagonists, or lower than the level or range in a control, for example, the level or range in a population of patients treated with a placebo, or lower than a specified threshold.

Allergic asthma is characterized by the production of type 2 cytokines, synthesis of immunoglobulin E (IgE), goblet cell metaplasia, influx of inflammatory cells and ultimately, airway remodelling. Example 2 demonstrates that mice with a restricted antibody repertoire do not develop allergic airway disease, and in particular, have an almost complete absence of the allergic airway disease seen in wild-type controls, including eosinophilia, recruitment and activation of pulmonary DCs, goblet cell hyperplasia, peribronchial and perivascular inflammatory cell infiltrates, lung pathology and the production of Th2-associated cytokines.

Importantly, Example 3 demonstrates that transfer of PCS-producing microbiota ameliorated eosinophilia, production of HDM-specific antibodies, lung pathology, goblet cell hyperplasia, mucus production, and secretion of Th2-associated cytokines.

Accordingly, in another embodiment the present invention provides a method as described herein wherein the administration of the therapeutically effective amount of the one or more eosinophil antagonist results in reduced goblet cell hyperplasia.

Accordingly, in another embodiment the present invention provides a method as described herein wherein the administration of the therapeutically effective amount of the one or more eosinophil antagonist results in reduced mucus production.

Accordingly, in another embodiment the present invention provides a method as described herein wherein the administration of the therapeutically effective amount of the one or more eosinophil antagonist results in reduced peribronchial and/or perivascular inflammatory cell infiltrate.

Accordingly, in another embodiment the present invention provides a method as described herein wherein the administration of the therapeutically effective amount of the one or more eosinophil antagonist results in reduced infiltration of neutrophils into the lungs.

In another embodiment the present invention provides a method as described herein wherein the administration of the therapeutically effective amount of the one or more eosinophil antagonist results in reduced pathologic change in the lungs.

In one embodiment, goblet cell hyperplasia and/or pathologic change are measured using histology.

In another embodiment the present invention provides a method as described herein wherein the administration of the therapeutically effective amount of the one or more eosinophil antagonist results in reduced production of Th2-associated cytokines.

In one embodiment, the level or range of production of one or more Th2-associated cytokines following administration of one or more eosinophil antagonists is at least about 5%, at least about 10%, at least about 15%, at least about 20%, at least about 25%, at least about 30%, at least about 35%, at least about 40%, at least about 45%, at least about 50%, at least about 55%, or at least 60%, at least about 65%, at least about 70%, at least about 75%, at least about 80%, at least about 85%, at least about 90%, at least about 95% compared to the level or range of the one or more Th2-associated cytokines prior to administration of the therapeutically effective amount of the one or more eosinophil antagonists, or lower than the level or range in a control, for example, the level or range in a population of patients treated with a placebo, or lower than a specified threshold.

In one embodiment, the Th2-associated cytokines are IL-5 and/or IL-13.

Example 3 demonstrates that transfer of PCS-producing microbiota ameliorated production of HDM-specific antibodies. Accordingly, in one embodiment, the present invention provides a method as described herein wherein the administration of the therapeutically effective amount of the one or more eosinophil antagonist results in reduced production of allergen-specific antibodies.

Individuals can become sensitised to allergens, wherein specific T- and B-lymphocytes are activated, leading to the production of allergen-specific antibodies, including immunoglobulin E (IgE).

As used herein the term “allergen-specific antibodies” refers to antibodies that bind specifically to an allergen.

In one embodiment the allergen is an aeroallergen.

In one embodiment, the present invention provides a method as described herein wherein the administration of the therapeutically effective amount of the one or more eosinophil antagonist results in reduced production of allergen-specific IgE.

As used herein the term “allergen-specific IgE” refers to immunoglobulin E (IgE) antibodies that bind specifically to an allergen, including those that bind to IgE receptors causing activation of cells, such as mast cells and basophils.

In one embodiment, the present invention provides a method as described herein wherein the administration of the therapeutically effective amount of the one or more eosinophil antagonist results in reduced production of house dust mite specific antibodies.

As used herein the term “house dust mite specific antibodies” refers to antibodies that bind specifically to a house dust mite allergen.

In one embodiment the allergen is selected from the group consisting of a house dust mite (e.g. Dermatophagoides sp.), a house dust mite derived molecule, and ligands derived from mite-associated bacterial and fungal products.

In one embodiment, the present invention provides a method as described herein wherein the administration of the therapeutically effective amount of the one or more eosinophil antagonist results in reduced production of house dust mite specific IgE.

An allergy is a disorder characterized by an allergic response to antigen, in particular, by the generation of antigen-specific IgE and the resultant effects of the IgE antibodies. As is well-known in the art, IgE binds to IgE receptors on mast cells and basophils. Upon later exposure to the antigen recognized by the IgE, the antigen cross-links the IgE on the mast cells and basophils causing degranulation of these cells.

In one embodiment, the level or range of production of antibodies (e.g. allergen-specific antibodies, allergen-specific IgE, house dust mite specific antibodies, or house dust mite specific IgE) following administration of one or more eosinophil antagonists is at least about 5%, at least about 10%, at least about 15%, at least about 20%, at least about 25%, at least about 30%, at least about 35%, at least about 40%, at least about 45%, at least about 50%, at least about 55%, or at least 60%, at least about 65%, at least about 70%, at least about 75%, at least about 80%, at least about 85%, at least about 90%, at least about 95% compared to the level or range of production of antibodies (e.g. allergen-specific antibodies, aeroallergen-specific antibodies, allergen-specific IgE, house dust mite specific antibodies, or house dust mite specific IgE) prior to administration of the therapeutically effective amount of the one or more eosinophil antagonists, or lower than the level or range in a control, for example, the level or range in a population of patients treated with a placebo, or lower than a specified threshold.

In normal health, CCR6 and CCL20 (the CCR6-CCL20 axis) perform an immune tolerance role by up-regulating immune suppression. When confronted with an inflammatory stimulus FoxP3+ regulatory Treg cells tend to proliferate aided by its cytokine milieu. If this typical homeostatic function is disrupted, it is known to result in a marked increase of the Th1/Th17 axis thereby promoting adverse immunologic function of multiple systems culminating in a number of diseases including sarcoidosis, idiopathic pulmonary fibrosis, chronic liver disease, experimental autoimmune encephalomyelitis, multiple sclerosis, rheumatoid arthritis, dry eye disease, psoriasis, glomerular nephritis, inflammatory bowel disease, HIV and an array of malignant cancers and their metastasis.

Example 6 demonstrates that PCS completely abrogated HDM-induced production of an airway epithelial cell-derived DC chemoattractant, CCL20 but did not have an effect on other chemokines, and that CCL20 levels were reduced in the BALF of L-tyrosine-treated mice exposed to HDM.

Accordingly, in one embodiment, the present invention provides a method as described herein, wherein the administration of the therapeutically effective amount of the one or more eosinophil antagonist results in reduced CCR6 signalling in the subject.

In another embodiment, the present invention provides a method as described herein, wherein the administration of the therapeutically effective amount of the one or more eosinophil antagonist results in reduced CCL20 expression in airway epithelia in the subject.

In a further embodiment the administration of the therapeutically effective amount of the one or more eosinophil antagonist results in reduced CCL20 secretion in airway epithelia in the subject.

In one embodiment, the subject administered the therapeutically effective amount of the one or more eosinophil antagonist has a disease or disorder associated with a dysregulation or an alteration of the CCR6-CCL20 axis.

In the lungs, CCR6 is co-expressed on alveolar macrophages in patients of sarcoidosis and alveolitis along with CXCR3 and CXCR6. CCR6+ T cells infiltrated into the lung interstitial tissue and were responsive to CCL20, CXCL10 and CXCL16. This observation demonstrates that T cells bearing CCR6 act in a coordinated manner with ligand and inflammatory cytokines produced by TH1 during alveolitic disease. Furthermore, CCR6 possesses the capability to recruit antigen-presenting immature and mature dendritic cells (DC) and macrophages to sites of inflammation on the alveolar epithelium.

In one embodiment, a subject administered the therapeutically effective amount of the one or more eosinophil antagonist has a disease or disorder associated with a dysregulation or an alteration of the CCR6-CCL20 axis in the lungs.

In the kidneys, glomerulonephritis is characterized by tissue damage caused due to T cell trafficking into the kidney. Chemokines modulate the migration of T lymphocytes to sites of inflammation. Renal FoxP3+ regulatory T cells (Treg) and IL-17 releasing TH17 cells were shown to upregulate CCR6 while IFN-γ releasing TH1 cells are CCR6 negative. Tregs and TH17 subsets displayed migratory capability towards CCL20 which is markedly high in renal biopsies of experimental murine nephritis. T cell recruitment is followed by pathogenesis in the kidney with albuminuria, leading to loss of renal function. Nephritic mice deficient in CCR6 demonstrated extreme renal damage and high mortality in comparison to the wild type, due to reduced accumulation of Treg cells and TH17 cells and not that of the TH1 type. Reintroduction of wild-type (WT) Treg provided protection to CCR6 knockout mice against severe renal injury, confirming that CCR6 promotes the recruitment of both TH17 and regulatory T reg cells to the kidney whereas a decrease in Tregs in the presence of TH1 response produced aggravated disease. Tregs have also been implicated in maintaining tolerance to autoimmune renal disease, thereby lowering renal inflammation, and in preventing allogenic responses in renal transplantation. CCR6 and CCL20 are reported to be involved in recruiting T and B cells to kidney nodules during chronic inflammation in individuals. Similar to CCR6-CCL20 acting as a mediator in the modelling of gut-associated lymphatic tissue, it is postulated that the nodular infiltrates in the kidney are also formed in a CCR6-dependent manner.

In one embodiment, a subject administered the therapeutically effective amount of the one or more eosinophil antagonist has a disease or disorder associated with a dysregulation or an alteration of the CCR6-CCL20 axis in the kidneys.

In the liver, chronic liver injury results from hepatic inflammation, leading to organ fibrosis. Intrahepatic increases in CCR6 and CCL20 expression have been observed in patients with chronic liver disease compared to healthy controls. It has been demonstrated that CCR6 and CCL20 contribute to the migration of gamma-delta (γδ) T cells, TH17 and regulatory (Treg) cells to sites of inflammation. CCR6 is explicitly required by IL-17 expressing γδ T cells to gather in the injured liver and promote disease resolution. Immunohistochemistry revealed accumulation of mononuclear cells bearing CCR6 induced by CCL20 secretion of hepatic parenchymal tissue in clinical liver disease. Compared to the WT, CCR6 knockout mice developed more acute fibrosis with enhanced immune cell infiltration to the liver.

In one embodiment, a subject administered the therapeutically effective amount of the one or more eosinophil antagonist has a disease or disorder associated with a dysregulation or an alteration of the CCR6-CCL20 axis in the liver.

In the brain, TH17 is strongly associated with autoimmune diseases, as demonstrated by pre-clinical studies in rheumatoid arthritis and multiple sclerosis. Neutralizing IL-17 as well as transfer of TH17 lacking CCR6 receptors had markedly inhibited experimental autoimmune encephalomyelitis (EAE). Apart from autoimmune promoting, pro-inflammatory function of TH17, it is also known to bring about disease resolution. Chemokines and adhesion molecules activate T cells, propelling them to migrate towards the central nervous system (CNS). The choroid plexus constitutively expresses CCL20 and acts as an entry point for CCR6 expressing CD4+ T cells. EAE in animal models is used to study multiple sclerosis, which is a demyelinating inflammatory disorder of the CNS and infiltrating T cells contribute to its pathogenesis. Effector TH17 and TH1 subsets are found in multiple sclerosis lesions along with the expression of cytokines IL-17 and IFN-γ. CCR6 demonstrates a critical aspect in the entry of TH17 which is said to induce EAE in the CNS. CNS-infiltrating cells, when analyzed directly for CCR6 expression, have revealed that in EAE, TH1 cells are in excess of TH17 CD4+ and both subtypes however, expressed CCR6. Cerebral ischemia or stroke is ranked the second globally most common cause of death and is a much-debilitating neurological disease condition. Immune-mediated tissue damage occurs in the first few days of suffering a stroke and is mainly attributed to brain-infiltrating, IL-17 releasing, γδ T cells which are largely positive for the chemokine receptor CCR6 as they trigger a highly conserved immune reaction. In a model of experimental stroke, genetic deficiency in CCR6 was associated with diminished infiltration of natural IL-17 releasing γδ T cells and a significantly improved neurological outcome, outlining the role CCR6 plays in pro-inflammatory immune cell chemotaxis to inflamed sites in the brain.

In one embodiment, a subject administered the therapeutically effective amount of the one or more eosinophil antagonist has a disease or disorder associated with dysregulation or an alteration of the CCR6-CCL20 axis in the brain.

In the eyes, TH17 cells are the principal effector cells causing inflammation in dry eye disease (DED), an immune inflammatory condition affecting the ocular surface that can even lead to corneal perforation. Local neutralization of CCL20 with antibodies administered sub-conjunctively to DED mice decreased TH17 cell permeation into the ocular surface producing improvement in clinical signs, indicating that CCR6 interaction with CCL20 directs the passage of TH17 cells in the eye. Inhibition of the CCR6/CCL20 axis is proposed to treat and/or prevent this condition.

In one embodiment, a subject administered the therapeutically effective amount of the one or more eosinophil antagonist has a disease or disorder associated with dysregulation or an alteration of the CCR6-CCL20 axis in the eye.

The skin disorder atopic dermatitis (AD) is identified by a deficiency of keratinocytes in the skin, which produces less CCL20, and a reduction in the expression of CCR6, which leaves patients exposed to viral infections leading to eczema herpeticum (ADEN) or eczema vaccinatum (EV). A population-based study of European and African descent had recorded single nucleotide polymorphism (SNP) in CCL20 in native Europeans significantly associated with AD, suggesting that variants in CCL20 and CCR6 are highly relevant to AD and increase the risk of severe viral complications in this skin disease. Psoriasis is a commonly occurring autoimmune skin disease that involves TH17 associated signalling pathways. CCR6 deficient mice fail to develop psoriasiform dermatitis in skin following IL-23 injections, because IL-23 is a growth and differentiation factor of TH17 cells and hence a typical driver of TH17 mediated inflammation. Previous research demonstrated that recombinant IL-23 injections into the skin of mice results in psoriasiform dermatitis that mimics human psoriasis in as short a period as 5 days. A more recent experimental model has documented that dermal CCR6+TH17 cells are sustained by IL-23 released from dendritic cells and these TH17 populations release IL-22 to stimulate epidermal hyperplasia through signal transducer and activator of transcription 3 (STAT3) mediated mechanisms in the human skin. Additionally, positive feedback was provided by epidermal and dermal production of CCL20, potentially recruiting more CCR6 expressing T cells or antigen presenting cells into inflamed psoriatic skin. Inhibition of CCR6 axis is proposed to treat and/or prevent this condition.

In one embodiment, a subject administered the therapeutically effective amount of the one or more eosinophil antagonist has a disease or disorder associated with dysregulation or an alteration of the CCR6-CCL20 axis in the skin.

Rheumatoid arthritis causes chronic inflammation of the joints where chemokines regulate infiltration of synovial fluid by inflammatory cells. This autoimmune disease is characterized by the increased release of CCL20 and the build-up of CCR6 bearing mononuclear T cells in the joints. An arthritis-induced study model of CCR6−/− mice had not exhibited any clinical signs consistent with disease compared to WT controls, but revealed that CD4+ T cells, TH17 cells and CD25 FoxP3+ regulatory T cells showed up-regulation of CCR6 with RANKL, which contributed towards disease, particularly osteoclastogenesis. A possible role in pathogenesis is thus highlighted in CCR6 in promoting inflammation at the joints. Ccr6 single nucleotide polymorphisms (SNPs) have demonstrated diminished basal and ligand induced Gαi protein signalling which predisposes individuals to diseases such as rheumatoid arthritis.

In one embodiment, a subject administered the therapeutically effective amount of the one or more eosinophil antagonist has a disease or disorder associated with dysregulation or an alteration of the CCR6-CCL20 axis in a joint.

Capacitated human sperm exhibits a directional movement towards CCL20 having the CCR6 receptor localized in the tail, and a recent study revealed modifications in motility parameters of spermatozoa in the presence of chemokines. In non-inflammatory conditions, chemokine receptor/ligand interactions within the reproductive tracts of the both sexes promote sperm motility and chemotaxis. Physiological reactions are thus mediated by CCR6 ligands in the male genitourinary system which extends beyond an inflammatory response. The present inventors propose the methods and compositions described herein are useful for modulating the CCR6-CCL20 axis in non-inflammatory conditions.

In one embodiment, a subject administered the therapeutically effective amount of the one or more eosinophil antagonist has a disease or disorder associated with dysregulation or an alteration of the CCR6-CCL20 axis in non-inflammatory conditions.

In the gut, animal models have identified: (i) genetic predisposition; (ii) the composition of associated microbiome; (iii) breakdown of innate immune barriers— disruption of the mucosal barrier due to decreased mucin synthesis, dysfunctional Toll-like and Nod-like receptor mediated pathways leading to increased pathogenicity, endoplasmic reticulum (ER) stress mediated apoptosis; (iv) deregulated adaptive immunity; and (v) a plethora of environmental factors, as multiple causes responsible for inflammatory disorders in the gastrointestinal tract (GI) and disruption of the CCR6/CCL20 axis, also as a significant contributing factor. Genome-wide association studies have confirmed Ccr6 as a risk allele of gastrointestinal tract infections, giving prominence to the CCR6/CCL20 axis as a potential risk factor which determines disease outcome. TH17 cells are directed to the small intestine by CCR6 upon immune induction and not only TH17, but also FoxP3+ regulatory Tregs are upregulated, given the fact that CCR6 performs dual functions with regards to these two helper T subsets in gut associated lymphoid tissue (GALT). Accumulation of TH17 cells in the spleen and bone marrow in CCR6 deficient mice showed they were unable to migrate due to the absence of this receptor, and hence produced less intestinal inflammation. This fact further supports its role in directing immune cell movement in the gut and confirms that TH17 plays a pro-inflammatory role in intestinal disorders. The intestinal microbiome is important for: (i) colonization and maintenance of immune cells; (ii) TH17—Treg balance in the gut; and (iii) protection against intestinal pathogens, evidenced by a reduction in TH17 and elevated Treg populations in mice given: (i) antibiotics; and (ii) bred in germ-free conditions. Disease outcome therefore primarily depends upon the CCR6-CCL20 axis, with microbiota featuring as another additional contributor. Inflammatory bowel disease (IBD), which is an autoimmune GI tract disorder, consists of two clinical variants, Crohn's disease and Ulcerative colitis. A Ccr6 knockout murine models had displayed: (i) smaller Peyer's patches; (ii) reduced sub epithelial domes; (iii) absence of isolated lymphoid follicles; (iv) reduced intestinal M cell numbers; (v) increased resistance to bacteria which enters through M cell conduits; (vi) marked elevation in the number of TH17 cells in the spleen and lymph nodes; (vii) Reduced migration to inflamed sites and less suppressive capabilities of Treg cells; (viii) moderate and severe disease in DSS and TNBS induced colitis respectively; and (ix) transfer of naïve T cells to Rag2−/− mice resulting in aggravated disease. SNPs in Ccr6 have been reported to predispose individuals to Crohn's disease.

In one embodiment, a subject administered the therapeutically effective amount of the one or more eosinophil antagonist has a disease or disorder associated with dysregulation or an alteration of the CCR6-CCL20 axis in the gastrointestinal tract.

Chemokines are utilized by cancer cells to directly invade the lymphatic system and spread via blood, as well as determine the location of metastatic growth of various tumors. CCL20 has been reportedly expressed in varied human cancer types, such as melanoma, adenocarcinoma, hepatocellular carcinoma leukemia, lymphoma, prostate cancer, colorectal, oral and lung squamous cell carcinoma and pancreatic carcinoma (PCA). The CCL20/CCR6 system has been demonstrated within pancreatic cancer cell lines and PCA-associated tissues. The stimulation of PCA cells expressing CCR6 with CCL20 had constitutively triggered cell proliferation, tendency to migrate and invasion of tissues indicating that CCL20 can act using mechanisms of autocrine and paracrine secretion. Recent studies have identified matrix metalloproteinase production to up-regulate CCL20, which promotes pancreatic tumor cell movement and their metastatic invasion. CCR6 inhibition in patients undergoing surgical treatment or clinical therapy has been proposed to be important to prevent liver metastasis of cancer, based on overexpression of functional CCR6 and CCR7 on metastatic tumor cell lines obtained from the liver. CCR6 directs and drives the mechanisms of chemotaxis, commonly adopted by malignant cancers when metastasizing to the liver. Mutations in Ccr6 also have been associated with a case of mucosa-associated lymphoid tissue (MALT) lymphoma.

In one embodiment, a subject administered the therapeutically effective amount of the one or more eosinophil antagonist has cancer or is at risk of developing cancer.

Preferential infection by HIV of CCR6+TH17 cells in vitro has been described in a study which using cultured TH1 and TH17 cells obtained from peripheral blood of healthy individuals in the presence of activated IL-1β and IL-23. Infection by HIV had produced negligible effects on TH1 whilst causing a significant reduction in TH17 cells, increased infection of TH17 cells and cell death. This study demonstrated a role for CCR6 in the internalizing of the virus within T helper populations. The CCR6/CCL20 axis is involved in actively recruiting TH17 cells and DCs to infection sites, thus helping the virus to propagate to other locations of the body. Envelope surface glycoprotein gp120 is known to significantly promote the CCR6 expression on human B cells.

In one embodiment, a subject administered the therapeutically effective amount of the one or more eosinophil antagonist has HIV or is at risk of acquiring HIV.

Minor inflammation of the adipose tissue has been linked with obesity and is driven via the CCR6-CCL20 axis. Adipose tissue lymphocytes expressing CCR6 have demonstrated chemotactic migration towards mature adipocytes which upregulate the chemokine ligand CCL20. Strongly enhanced CCL20 expression by adipocytes displayed a positive correlation with the body mass index (BMI), in visceral adipose tissue compared to the subcutaneous fat layers. Increased leukocyte streaming into pancreatic islets causes inflammation in the beta cell mass influencing apoptosis and dysfunction. Elevation of CCL20 levels in pancreatic beta cells induced by the transcription factor nuclear factor kappa B (NF-kB) has been demonstrated. T cell immunity is suppressed by the activation of type III histone deacetylase Sirtuin 1, which is known to regulate cellular processes via the SIRT1 gene. Resveratrol is a Sirtuin-1 activator which demonstrated therapeutic efficacy in a Nucleotide-binding and oligomerization domain (NOD) mouse model of type 1 diabetes. Resveratrol-treated mice exhibited a significant decrease in Ccr6 in a gene array analysis, correlating with decreased migration in CCR6+macrophages and IL-17 producing cells into the pancreas from pancreatic lymph nodes.

In one embodiment, a subject administered the therapeutically effective amount of the one or more eosinophil antagonist is overweight, has diabetes, obesity or metabolic syndrome.

In one embodiment, the level or range of expression of CCL20 (e.g. CCL20 secretion in airway epithelia in the subject) following administration of one or more eosinophil antagonists is at least about 5%, at least about 10%, at least about 15%, at least about 20%, at least about 25%, at least about 30%, at least about 35%, at least about 40%, at least about 45%, at least about 50%, at least about 55%, or at least 60%, at least about 65%, at least about 70%, at least about 75%, at least about 80%, at least about 85%, at least about 90%, at least about 95% compared to the level or range of expression of CCL20 (e.g. CCL20 secretion in airway epithelia in the subject) prior to administration of the therapeutically effective amount of the one or more eosinophil antagonists, or lower than the level or range in a control, for example, the level or range in a population of patients treated with a placebo, or lower than a specified threshold.

In one embodiment, the level or range of CCR6 signalling following administration of one or more eosinophil antagonists is at least about 5%, at least about 10%, at least about 15%, at least about 20%, at least about 25%, at least about 30%, at least about 35%, at least about 40%, at least about 45%, at least about 50%, at least about 55%, or at least 60%, at least about 65%, at least about 70%, at least about 75%, at least about 80%, at least about 85%, at least about 90%, at least about 95% compared to the level or range of CCR6 signalling prior to administration of the therapeutically effective amount of the one or more eosinophil antagonists, or lower than the level or range in a control, for example, the level or range in a population of patients treated with a placebo, or lower than a specified threshold.

CCL20—Chemokine (C-C motif) ligand 20—is also commonly referred to as macrophage inflammatory protein 3-alpha (MIP-3cc) or liver activation regulated chemokine (LARC). CCL20 functions normally as a chemotactic factor for the recruitment of T-, B-, and immature dendritic-cells, and is produced predominantly by cells of the liver, lung, and gastrointestinal tract. Chemokine receptor 6 (CCR6) has been identified as the receptor for CCL20 and, to date, is still the lone functional receptor identified for the CCL20 ligand.

CCR6 is sometimes also referred to as CD 196 or CD 196 antigen. Other terms for CCR6 including for example, CC-CKR-6, C-C-CKR-6, Chemokine (C-C Motif) Receptor 6, Chemokine (C-C) Receptor 6, C-C Chemokine Receptor Type 6, CKRL3, CKR-L3, Chemokine Receptor-Like 3, STRL22, CMKBR6, G Protein-Coupled Receptor 29, GPR29, Seven-Transmembrane Receptor, Lymphocyte 22, GPRCY4, GPR-CY4, DRY6, LARC Receptor, and BN-1.

One of ordinary skill in the art will be able to identify the polynucleotide and amino acid sequences of CCR6 receptor and CCL20, as well as any orthologous and splice variant isoforms of CCR6 and CCL20 from any sequence database (e.g. the NCBI database), including human sequences.

Sensing of a common allergen, house dust mite via Toll-like receptor 4 (TLR4) expressed on airway epithelial cells has been shown to be necessary for the activation of pulmonary DCs and the initiation of allergic sensitization.

Example 6 demonstrates that PCS abrogates HDM-induced and LPS induced production of CCL20, and reduces TLR4 signalling via LPS. Accordingly, in one embodiment, the present invention provides a method as described herein, wherein the administration of the therapeutically effective amount of the one or more eosinophil antagonist results in reduced TLR4 signalling in the subject.

Several pathogen-associated molecular patterns (PAMPs) can stimulate TLR4. These molecules include lipopolysaccharide (LPS) from Gram-negative bacteria, fusion (F) protein from respiratory syncytial virus (RSV) and the envelope protein from mouse mammary tumor virus (MMTV). In addition, endogenous molecules can also interact directly or indirectly with TLR4, such as heat-shock proteins, hyaluronic acid and β-defensin 2. LPS stimulation of mammalian cells occurs through a series of interactions with several proteins including the LPS binding protein (LBP), CD14, MD-2 and TLR4. LBP is a soluble shuttle protein which directly binds to LPS and facilitates the association between LPS and CD14. CD14 is a glycosylphosphatidylinositol-anchored protein, which also exists in a soluble form. CD14 facilitates the transfer of LPS to the TLR4/MD-2 receptor complex and modulates LPS recognition.

As used herein the term “reducing TLR4 signalling” includes a reduction in the activation of at least one downstream signalling pathway which has resulted from the activation of TLR4, for example in response to LPS or another PAMP. Typically, the signalling is an intracellular signalling cascade which is initiated by the TIR domain of TLR4. The signalling cascade induced by TLR4 may result in activation of the transcription factors such as NF-KB, or interferon regulated factor 3. TLR4 mediated signalling may further activate mitogen-activated protein kinases (MAPKs), p38, c-jun, N terminal kinase (JNK) and p42/44.

In one embodiment the TLR4 signalling may be activated by a PAMP leading to a cytokine response. The TLR4 signalling protein activated may be one or more of NFκB, IκBα, IRF3, p38 and p42/44.

Accordingly, in one embodiment the present invention provides methods for treating and preventing gram negative bacterial infection, sepsis, septic shock and/or inflammation associated with LPS.

In one embodiment the PAMP may be a gram negative bacterium or a gram negative bacterial component such as LPS.

In one embodiment, a subject administered the therapeutically effective amount of the one or more eosinophil antagonist has a gram negative bacterial infection or is at risk of acquiring a gram negative bacterial infection.

In one embodiment, a subject administered the therapeutically effective amount of the one or more eosinophil antagonist has sepsis or is at risk of developing sepsis.

TLR4 expression can be detected on many tumour cells and cell lines, and the link between TLR signalling and tumorigenesis is discussed in Korneev et al. (2017) Cytokine 89:127-135.

In one embodiment, a subject administered the therapeutically effective amount of the one or more eosinophil antagonist has cancer or is at risk of developing cancer.

Activation of TLR4 leads to downstream release of inflammatory modulators including TNF-α and Interleukin-1, and constant low-level release of these modulators has been proposed to reduce the efficacy of opioid drug treatment with time, and be involved in both the development of tolerance to opioid analgesic drugs. Accordingly, in one embodiment the present invention provides methods for reducing tolerance to an opioid and/or increase the analgesic effect of an opioid

In one embodiment, a subject administered the therapeutically effective amount of the one or more eosinophil antagonist is on opioid treatment.

In one embodiment, the level or range of TLR4 signalling following administration of one or more eosinophil antagonists is at least about 5%, at least about 10%, at least about 15%, at least about 20%, at least about 25%, at least about 30%, at least about 35%, at least about 40%, at least about 45%, at least about 50%, at least about 55%, or at least 60%, at least about 65%, at least about 70%, at least about 75%, at least about 80%, at least about 85%, at least about 90%, at least about 95% compared to the level or range of TLR4 signalling prior to administration of the therapeutically effective amount of the one or more eosinophil antagonists, or lower than the level or range in a control, for example, the level or range in a population of patients treated with a placebo, or lower than a specified threshold.

In another aspect, the present invention provides a method of reducing eosinophilia in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In one embodiment, the level or range of eosinophilia following administration of one or more eosinophil antagonists is reduced compared to the level or range of expression of eosinophilia prior to administration of the therapeutically effective amount of the one or more eosinophil antagonists, or lower than the level or range in a control, for example, the level or range in a patient or a population of patients treated with a placebo, or lower than a specified threshold.

In one embodiment, the subject has asthma, allergic airway disease, house dust mite associated allergic airway disease allergic rhinitis, and/or chronic rhinosinusitis.

In another aspect, the present invention provides a method of reducing infiltration of pulmonary dendritic cells into the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In another aspect, the present invention provides a method of reducing activation of pulmonary dendritic cells in the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In another aspect, the present invention provides a method of reducing migration of pulmonary dendritic cells into lymph nodes of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In another aspect, the present invention provides a method of reducing goblet cell hyperplasia in the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In another aspect, the present invention provides a method of reducing pathologic change in the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In another aspect, the present invention provides a method of reducing mucus production in the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In another aspect, the present invention provides a method of reducing a peribronchial and/or perivascular inflammatory cell infiltrate in the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In another aspect, the present invention provides a method of reducing infiltration of neutrophils into the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In another aspect, the present invention provides a method of reducing Th2-associated cytokine production in the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In one embodiment the Th2-associated cytokine is IL-5 and/or IL-13.

In another aspect, the present invention provides a method of reducing the production of allergen specific antibodies in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In another aspect, the present invention provides a method of reducing the production of house dust mite specific antibodies in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In one embodiment the antibodies are IgE antibodies.

In another aspect, the present invention provides a method of reducing the priming of T cells by pulmonary dendritic cells in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In another aspect, the present invention provides a method of reducing CCL20 expression in airway epithelia in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

The present inventors have demonstrated that CCR6 signalling can be reduced using a therapeutically effective amount of one or more eosinophil antagonist.

As discussed above, a plethora of research studies have demonstrated that the CCR6 and CCL20 axis directly influences the nervous, respiratory, gastrointestinal, excretory, skeletal, and reproductive systems via pleiotropic immune mechanisms, manifesting as diseases with high mortality rates. CCR6 is naturally expressed in multiple tissues: maximally in the appendix, spleen, lymph nodes and pancreas and minimally in the thymus, colon, small intestine, fetal liver and testis. CCR6 is upregulated by numerous leukocyte cohorts, such as B-cells, T-cells (specifically pro-inflammatory TH17 cells and immune regulatory Treg cells), immature dendritic cells, NKT cells, innate lymphoid cell 3 (ILC3) and neutrophils. The dominant role of CCR6 in inflammatory disease is underpinned by its influence on driving the T helper subset differentiation and maintaining leukocyte homeostasis. Naïve T helper cells resident in lymph nodes, upon antigen sampling will differentiate into its effector sub populations, TH17 and regulatory Treg cells, TH1 and TH2, mediated by the prevailing cytokine environment and a host of other factors. However, a critical factor which determines the development of TH17 and Treg subsets evidently becomes the upregulation of CCR6 as both these cell sub types are known to be CCR6+CD4+ T cells. Thus proliferation, migration and promoting pro- or anti-inflammatory effects of these helper sets might be primarily CCR6 dependent processes.

In one aspect, the present invention provides a method of reducing CCR6 signalling in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

Example 6 demonstrates that PCS abrogates HDM-induced and LPS induced production of CCL20, and reduces TLR4 signalling via LPS. Importantly, the mechanism of action of PCS is linked to its capacity to uncouple TLR-4—EGFR cross-talk, known to synergize for optimal signal transduction.

Accordingly, in one embodiment the present invention provides methods of reducing EGFR mediated signalling in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In one embodiment, the EGFR mediated signalling is EGFR mediated TLR4 signalling.

In another embodiment, the EGFR mediated signalling is EGFR mediated TLR4 signalling in response to LPS.

In one embodiment EGFR-TLR4 cross talk is reduced.

As used herein the term “EGFR-TLR4” cross talk refers to signalling via EGFR resulting from TLR-4 signalling from LPS.

In one embodiment the present invention provides a method of reducing LPS-induced septic shock in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In one embodiment, the level or range of EGFR mediated signalling following administration of one or more eosinophil antagonists is at least about 5%, at least about 10%, at least about 15%, at least about 20%, at least about 25%, at least about 30%, at least about 35%, at least about 40%, at least about 45%, at least about 50%, at least about 55%, or at least 60%, at least about 65%, at least about 70%, at least about 75%, at least about 80%, at least about 85%, at least about 90%, at least about 95% compared to the level or range of EGFR mediated signalling prior to administration of the therapeutically effective amount of the one or more eosinophil antagonists, or lower than the level or range in a control, for example, the level or range in a population of patients treated with a placebo, or lower than a specified threshold.

Example 3 demonstrates that transfer of PCS producing microbiota ameliorated allergic responses, including eosinophilia, production of HDM-specific antibodies, lung pathology, goblet cell hyperplasia, mucus production, and secretion of Th2-associated cytokines. Without wishing to be bound by theory, the present inventors propose that administration of L-tyrosine, N-acetyl-L-tyrosine, 4-hydroxyphenylpyruvate, 4-hydroxyphenylacrylate, 3-(p-hydroxyphenyl)propionate can lead to the production of 4-hydroxyphenylpyruvate, 4-hydroxyphenylacrylate, 3-(p-hydroxyphenyl)propionate, p-cresol, p-cresol glucuronide and/or p-cresol sulphate in the gut of the subject by intestinal bacteria. L-tyrosine and L-DOPA can also be produced from L-phenylalanine and L-tyrosine, respectively, in the subject.

Accordingly, in one embodiment the present invention provides a method as described herein, wherein L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and/or p-cresol sulphate is produced in the subject following administration of the one or more eosinophil antagonist.

The formulation, dosage regimen, and route of administration of one or more eosinophil antagonist, can be adjusted to provide an effective amount of the one or more eosinophil antagonist to have the desired result.

In one example, the one or more eosinophil antagonist is administered in an amount sufficient to have one or more of the following effects in the subject:

reducing eosinophilia;

reducing infiltration of pulmonary dendritic cells into the lungs of the subject;

reducing activation of pulmonary dendritic cells in the lungs of the subject;

reducing migration of pulmonary dendritic cells into lymph nodes of the subject;

reducing goblet cell hyperplasia in the lungs of the subject;

reducing pathologic change in the lungs of the subject;

reducing Th2-associated cytokine production in the lungs of the subject;

reducing the production of allergen specific antibodies in a subject

reducing the production of house dust mite specific antibodies in a subject;

reducing the priming of T cells by pulmonary dendritic cells in a subject;

reducing CCL20 expression in airway epithelia in a subject;

reducing CCR6 signalling in a subject;

reducing TLR4 signalling in a subject;

reducing EGFR mediated signalling in a subject;

reducing LPS-induced septic shock in a subject; and/or

improving a clinical measure of a disease or a disorder.

Clinical measures of a disease or a disorder are known in the field.

For example, in one embodiment improving a clinical measure of asthma includes:

reducing Acute Exacerbation Rate;

increasing Forced Expiratory Volume in one second results;

improving Asthma Control Questionnaire, 6-item version, results; and/or

improved Asthma Quality of Life Questionnaire results.

Suitable dosages of the one or more eosinophil antagonist of the present invention will vary depending on the antagonist, disease, disorder and/or the subject being treated. It is within the ability of a skilled person to determine a suitable dosage, e.g., by commencing with a sub-optimal dosage and incrementally modifying the dosage to determine an optimal or useful dosage. Alternatively, to determine an appropriate dosage for treatment, data from cell culture assays or animal models can be used, wherein a suitable dose is within a range of circulating concentrations that include the ED₅₀ of the active antagonist with little or no toxicity. The dosage may vary within this range depending upon the dosage form employed and the route of administration utilized. A therapeutically effective dose can be estimated initially from cell culture assays. A dose may be formulated in animal models to achieve a circulating plasma concentration range that includes the IC₅₀ (i.e., the concentration of the antagonist which achieves a half-maximal inhibition of symptoms) as determined in cell culture. Such information can be used to more accurately determine useful doses in humans.

In one aspect, the therapeutically effective amount of the one or more eosinophil antagonist is administered in one or more fixed doses. For example, in one embodiment the methods comprise administering to the subject one or more eosinophil antagonist in an effective amount and/or at sufficient interval to achieve and/or maintain a certain dose of the one or more eosinophil antagonist per volume of serum, using, for example, an assay as described herein.

Accordingly, in one embodiment the present invention provides a method as described herein, wherein the therapeutically effective amount of the one or more eosinophil antagonist is administered in two or more doses. For example, in 2, 3, 4, 5, 6, 7, 8, 9, 10 doses.

In another aspect, the therapeutically effective amount of the one or more eosinophil antagonist is administered at a regular interval, for example daily, twice daily, weekly, biweekly, monthly, bimonthly, or quarterly.

In one aspect, the therapeutically effective amount of the one or more eosinophil antagonist is administered at the regular interval over days, weeks, months, years or decades.

In another aspect, the subject administered with a therapeutically effective amount of the one or more eosinophil antagonist is treated before, during, after, or simultaneously with one or more additional therapies for the treatment of the eosinophilic disease or disorder.

In another aspect, the subject administered with a therapeutically effective amount of the one or more eosinophil antagonist is treated before, during, after, or simultaneously with one or more additional therapies for the treatment of the eosinophilic disease or disorder, a disease or disorder associated with CCR6 signalling, TLR4 signalling and/or EGFR mediated signalling.

In another aspect, the subject administered a therapeutically effective amount of the one or more eosinophil antagonist has received one or more additional therapies for the treatment of the eosinophilic disease or disorder.

In another aspect, the subject administered a therapeutically effective amount of the one or more eosinophil antagonist has received one or more additional therapies for the treatment of the eosinophilic disease or disorder, a disease or disorder associated with CCR6 signalling, TLR4 signalling and/or EGFR mediated signalling.

In another aspect, the subject is treated with, or has received, at least one therapeutically effective dose of oral or inhaled corticosteroids.

In one aspect, the therapeutically effective amount of the one or more eosinophil antagonist is administered in combination with another compound useful for treating a disease or condition described herein, either as combined or additional treatment steps or as additional components of a therapeutic formulation (e.g. a composition or a pharmaceutical composition).

In one aspect, the compound is a compound used to treat a hypereosinophilic syndrome, eosinophilic gastritis, eosinophilic gastroenteritis, eosinophilic esophagitis, eosinophilic pneumonia, eosinophilic granulomatosis with polyangiitis, allergy, dermatitis, asthma and chronic rhinosinusitis.

In one aspect, the present invention provides administering to a subject one or more intestinal bacteria capable of maintaining an effective amount of the one or more eosinophil antagonist described herein in the subject. For example, in one embodiment the present invention provides administering to a subject one or more bacterial strains capable of, including genetically engineered to be capable of, maintaining an effective amount per volume of serum, using, for example, an assay as described herein, of the one or more eosinophil antagonist in the subject.

The one or more eosinophil antagonist may be administered through any suitable means, compositions and routes known in the art.

In one embodiment, the therapeutically effective amount of the one or more eosinophil antagonist is administered orally, by inhalation, intravenously, intramuscularly, subcutaneously, topically or a combination thereof or any suitable means.

Preferably, the therapeutically effective amount of the one or more eosinophil antagonist is administered orally.

In another embodiment, the one or more eosinophil antagonist is formulated as a composition further comprising one or more physiologically acceptable carrier, excipient or diluent.

In one embodiment, the one or more eosinophil antagonist is formulated as a composition further comprising one or more physiologically acceptable carrier, excipient or diluent, and pectin and/or alginate.

In another embodiment, the one or more eosinophil antagonist is formulated as a composition further comprising one or more physiologically acceptable carrier, excipient or diluent, and one or more physiologically active agent for combination therapy.

In one embodiment, L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate can be provided in any physiologically acceptable salt.

In another embodiment, the present invention provides an oral dosage form or formulation comprising a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In another embodiment, the present invention provides an oral dosage form or formulation suitable for oral supplementation, for example, in the form of an oral supplement tablet, or an oral supplement powder.

For example, in one embodiment the present invention provides an oral dosage form or formulation suitable for oral administration according to the methods as described herein.

In one embodiment, the oral dosage form or formulation is an enterically coated oral dosage form.

In one embodiment, the oral dosage form or formulation is an infant food or infant formula comprising a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In one embodiment, the infant formula or food is additionally formulated with other nutritionally beneficial ingredients known in the art, e.g., oils providing longer chain polyunsaturated fatty acids, such as arachidonic acid and docosahexaenoic acid, vitamins, minerals, selenium, natural carotenoids, nucleotides, taurine and/or other nutrients.

In another embodiment the infant formula or food is a nutritionally complete infant formula or food.

In another embodiment, the infant food or infant formula is produced as a liquid product, a concentrated liquid product requiring dilution before administration, or a powder requiring formulating before administration.

In another embodiment, the composition is a pharmaceutical composition comprising a therapeutically effective amount of one or more eosinophil antagonist selected from L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In one aspect, the present invention provides a composition as described herein for use in the treatment and/or prevention of a pulmonary disease in a subject, wherein the composition comprises one or more eosinophil antagonist is selected from L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In another aspect, the present invention provides kits containing one or more eosinophil antagonist.

In one aspect, the present invention provides a method as described herein, or a composition as described herein, wherein the composition consists of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

In one aspect, the present invention provides a use of L-phenylalanine, L-tyrosine, N-acetyl-L-tyrosine, L-DOPA, 4-hydroxyphenylpyruvate, 4-hydroxyphenylacrylate, 3-(p-hydroxyphenyl)propionate, p-cresol, p-cresol glucuronide and p-cresol sulphate in the manufacture of a medicament for treating an eosinophilic disease or disorder in a subject. Preferably, the eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

### EXAMPLES

### Example 1: Materials and Methods

Experimental Animals

MD4 mice (on C57BL/6J background) were originally obtained from the Institute for Research in Biomedicine in Bellinzona, Switzerland or re-derived at Monash Animal Research Platform at Clayton, Victoria, Australia. C57BL/6J WT mice were originally obtained from Charles River Laboratories (L'Arbresle, France) or Monash Animal Research Platform (Clayton, Victoria, Australia). All mice were bred and maintained under specific pathogen-free conditions. JhT−/− mice (on C57BL/6J background) were obtained from École Polytechnique Fédérale de Lausanne (EPFL), Switzerland. OT-II mice were obtained from Monash Animal Research Platform. All mice were bred and maintained under specific pathogen-free conditions and fed irradiated WEHI Mice Cubes (Barastoc, product code 8720610). Heterozygous breedings of MD4 mice were set up using WT females mated with MD4 males. Given allelic exclusion in B cells and the strong promotor in the HyHEL10 construct in MD4 cells, this breeding strategy is the only way to generate both MD4 and WT littermate controls. Germ-free mice (C57BL/6J background) were obtained from the Clean Mouse Facility (CMF), University of Bern, Bern, Switzerland. 6-12 weeks mice were used for all experiments, except for L-tyrosine and p-cresol sulfate treatments, which were initiated at the age of 3 weeks. All animal experiments were performed in accordance with institutional guidelines, Swiss federal and cantonal laws on animal protection or approved by Monash Animal Ethics Committee.

L-Tyrosine/PCS Treatments In Vivo

Mice received water as a control or L-tyrosine (reagent grade, ≥98% Sigma-Aldrich, St. Louis, Mo.) resuspended in drinking water under sterile conditions at the concentration of 100 mg/kg/day or 500 mg/kg/day (based on the assumption of a mouse consuming 4 ml of water daily). All mice received the treatment 2 weeks prior and throughout the experiment. P-cresol sulfate (Alsachim, Illkirch-Graffenstaden, France) was resuspended in saline under sterile conditions and delivered via injection into the right retro-orbital sinus at the dose of 40 mg/kg in a volume of 200 μl one day prior to first HDM exposure (day −1) and 4 hours prior to second HDM exposure (day 11). Control mice received 200 μl of saline. Control mice received 200 μl of saline. For experiments employing antibiotic treatment, 3-week old mice were treated with a combination enrofloxacin (10 mg/kg/day) and amoxicillin with clavulanic acid (1 mg/kg/day) for one week, followed by one week of only amoxicillin with clavulanic acid (1 mg/kg/day). Then, mice were then put on L-tyrosine diet in drinking water for two weeks followed by HDM exposure as before. During this time, mice were maintained on antibiotic treatment with amoxicillin/clavulanic acid until end of experiment.

Animal Model of Allergic Airway Inflammation

Mice were anaesthetised by the inhalation of 4% isoflurane in oxygen for 3-5 minutes. 20 μg of protein content of crude house dust mite extract (HDM) (Greer Laboratories Inc., Lenoir, N.C.) in 20 μl of sterile phosphate-buffered saline (PBS) (Gibco™) was applied intranasally on days 0, 11, 12 and 13. Mice were humanely sacrificed with the lethal dose of pentobarbital (Streuli Pharma AG, Uznach, Switzerland) on day 14.

Animal Model of Pulmonary Type I Inflammation

Mice were anaesthetized by the inhalation of 4% isoflurane in oxygen for 3-5 min. 100 μg Ovalbumin (Invivogen, cat nr vac-pova) was mixed with 10 μg LPS from E. coli (Sigma-Aldrich, cat nr L4391) and administered intranasally per mouse on days 0, 11, 12 and 13. Mice were sacrificed with the lethal dose of pentobarbital on day 14.

Cellular Infiltration of the Airways

Bronchoalveolar lavage fluid (BALF) was collected by flushing airways with 0.5 ml PBS supplemented with 0.2% bovine serum albumin (Sigma-Aldrich, St. Louis, Mo.). Total cell number was determined with Coulter Counter (IG Instrumenten-Gesellschaft AG, Basel, Switzerland) while differential cell staining performed on cytospins stained with Diff-Quik solution (Dade Behring, Siemens Healthcare Diagnostics, Deerfield, Ill.). Percentages of neutrophils, macrophages, lymphocytes and eosinophils were assessed by counting 200 cells per sample.

ELISA

Concentrations of interleukin-4, -5, -13, -17 and IFN-γ in culture supernatants were analysed with mouse Ready-Set-Go!™ ELISA kits (eBioscience™, San Diego, Calif.) according to manufacturer's instructions. To measure the levels of HDM-specific IgG1 and IgE in plasma, half area 96-well plates (Corning) were coated with HDM (10 μg in PBS) overnight at 4° C., followed by the incubation of samples for 2 hours at RT, and the addition of alkaline-phophatase-conjugated goat anti-mouse IgE or IgG1 (both from SouthernBiotech, Birmingham, Ala.; diluted at 1 μg ml-1 in PBS 0.2% BSA) for 2 hours at RT. 4-nitrophenyl phosphate sodium salt hexahydrate (pNPP) (Sigma) was used as a substrate, and the colorimetric reaction was read at 405 nm on the Synergy H1 microplate reader (Biotek, Luzern, Switzerland). To measure the levels of total IgA and IgM as well as hen egg lysozyme (HEL)-specific IgA and IgM in mouse faeces, faecal pellets were homogenized in 0.8 ml cold PBS, centrifuged at 400 g for 5 minutes to remove large debris, filtered through 40 μm cell strainer and centrifuged at 8000 g for 10 minutes to pellet bacteria. The supernatant was collected and loaded for 2 hours at RT on 96-well half area plates coated a day before at 4° C. with anti-IgA (Southern Biotech; 2 μg ml-1), anti-IgM (SouthernBiotech; 2 μg ml-1) or HEL protein (Sigma-Aldrich; 10 μg/ml). This step was followed by the addition of alkalinephophatase-conjugated goat anti-mouse IgA or IgM (both at 1 μg ml-1 in PBS 0.2% BSA) for 2 hours at RT. 4-nitrophenyl phosphate sodium salt hexahydrate (pNPP) (Sigma) was used as a substrate, and the colorimetric reaction was read at 405 nm as before.

Tyrosine Assay

Faeces were collected freshly, homogenized in distilled water and centrifuged 8000 g for 5 min at 4° C. Supernatant was filtered through a 40 μM cell strainer and deproteinized using 10 kDa spin columns (Abcam). L-tyrosine concentrations were measured with Tyrosine assay kit (Abcam) according to manufacturer's instructions.

Intestinal Permeability Assay

Mice were water starved overnight and FITC-dextran administered by oral gavage at 0.44 mg/g body weight. 6 h later mice were sacrificed, blood collected and FITC-dextran concentrations measured via fluorescence spectrophotometry.

Kidney Toxicity Markers

Concentrations of cystatin, clusterin, lipocalin-2 and osteopontin in serum of vehicle or PCStreated mice were determined with MILLIPLEX MAP Mouse Kidney Injury Magnetic Bead Panel 2—Toxicity Multiplex Assay (Merck) according to manufacturer's instructions.

Flow Cytometry

Mediastinal lymph nodes were filtered through a 40 μm cell strainer, washed with PBS supplemented with 1% fetal bovine serum and 2 mM EDTA (Invitrogen) (MACS buffer). Lungs were finely cut with scissors, digested with Collagenase IV (Gibco™) in Iscove's modified Dulbecco's medium (IMDM, Gibco™) for 50 min at 37° C. and processed as the lymph nodes. Cell counts were determined with Coulter Counter and 105 cells were stained with freshly prepared antibody mix in MACS buffer for 20 minutes at 4° C. in a 96-well round-bottom plates (Costar). Dendritic cells were identified using monoclonal antibodies against CD11c-phycoerythrin (PE)/Cy7 (Biolegend, cat nr 117318; diluted 1:400 in MACS buffer), SiglecF-Alexa Fluor (AF) 647 (BD Biosciences™, 562680; 1:400) and MHC-II-AF700 (Biolegend, 107622, 1:800). DC activation was assessed using antibodies against PD-L2-PE (Biolegend, 107205; 1:200), CD80− Brilliant Violet (BV)-605 (Biolegend, 104729; 1:200) and CD86-BV650 (Biolegend, 105035; 1:200). T helper cells were identified using antibodies against CD3c-Pacific Blue (PB) (Biolegend, 100214; 1:800) and CD4− PerCP-Cy5.5 (Biolegend, 100434; 1:800). Activated T helper cells were identified using anti-CD44-PE antibody (BD Biosciences™, 553134, 1: 400) A regulatory subset of T helper cells (Tregs) was identified with the addition of anti-CD25-AF700 (Biolegend, 102024; 1:200) and anti-Foxp3-AF647 (Biolegend, 126408; 1:200) antibodies. For the latter, an intracellular staining was performed, where cells were incubated with anti-Foxp3 antibody diluted in 0.5% saponin from Quillaja bark (Sigma-Aldrich) for 40 minutes at 4° C. B cells were identified with anti-CD19-PE/Cy7 (eBioscience, 25-0193; 1:200) and anti-B220-FITC (Biolegend, 103206; 1:200). When indicated, HEL and HDM were labelled with Alexa Fluor 647 antibody labelling kit (Invitrogen™) and separated from the unlabelled dye with the use of PD-10 desalting columns (GE Healthcare). Both HEL-AF647 and HDM-AF647 were used for extracellular staining in a dilution of 1:200 in MACS buffer. Cells were acquired on BD Fortessa (BD Biosciences™, San Jose, Calif.). Samples were analyzed with FlowJo 10.4.2 software (Tree Star Inc., Ashland, Oreg.).

Histology

Right lung lobes were fixed in 10 ml of 10% buffered formalin at 4° C. and embedded into paraffin. Prepared sections (4 μm) were stained with either H&E or PAS reagents using standardized protocols and analyzed with an Axioskop 2 plus microscope equipped with an Axio-Cam HRc (Carl Zeiss Microimaging GMbH, Jena, Germany).

In Vivo Tracking of DCs

Mice were administered 20 μg HDM-AF647 in 20 μl PBS intranasally. Dendritic cell antigen uptake, activation and migration to lung-draining lymph nodes were performed by flow cytometry.

Ex-Vivo Restimulation Assay

Mediastinal lymph node suspensions were filtered through 40 μm cell strainer, washed and resuspended in IMDM medium supplemented with 10% fetal bovine serum, 1% Penicillin/Streptomycin (Invitrogen™), 0.05 mM 2-mercaptoethanol (Gibco™). Cells were plated in a 96-well round-bottom culture plates (Costar) at the density of 105 cells/well in the presence of HDM (0-50 μg/ml, based on protein content) and cultured for 4 days at 37° C. 5% CO2, after which the supernatants were collected for cytokine quantification.

DC: T cell co-cultures were set-up by sorting CD11c+ SiglecF− (DCs) and CD4+CD44+ T cells from the lungs of HDM-immunized mice on FACSAria III (BD Biosciences™, San Jose, Calif.). 5000 DCs and 10000 T cells were plated per well in a 96-well round-bottom culture plates and stimulated with HDM (40 μg/ml) for 4 days, after which the supernatants were collected.

Bacterial Cell Sorting

Fresh faeces were homogenized in ice cold PBS, filtered through 40-μM cell strainers and centrifuged at 400 g for 5 minutes at 4° C. Supernatant was collected, diluted 3× with PBS 1% BSA and centrifuged at 400 g for 5 min at 4° C. This step was repeated twice to remove debris and mammalian cells. Bacterial cells were spun down at 8000 g for 5 min at 4° C., and stained with SYTO BC (1:8000) for 30 min at 4° C. They were subsequently blocked with 20% normal rat serum for 20 min at 4° C. and stained with anti-IgA-PE (1:200, cat nr 12-4204-82, eBioscience) for 20 min at 4° C. Cells were then incubated with anti-PE beads (Miltenyi) and enriched by MACS. MACS-sorted cells were stained with anti-IgA-AF647 (1:100, cat nr 1040-31, SouthernBiotech) for 20 min at 4° C. Final bacterial cell populations were sorted by FACSAria III as SYTO BC+PE+AF647+(IgA-positive) or SYTO BC+PE−AF647− (IgA-negative).

Bacterial DNA Isolation from Mouse Faeces

One faecal pellet from each mouse was collected into sterile 1.5 ml Biopure tube (Eppendorf, Hamburg, Germany), put immediately on dry ice and stored at −80° C. until further processing. Total bacterial DNA was isolated using the QiaAMP Fast DNA Stool Mini Kit (QIAGEN) according to manufacturer's instructions. DNA was eluted with 100 μl of AE buffer (provided with the kit). DNA was stored at 4° C. until being used for the PCR.

Bacterial DNA Isolation from Faecal Pellets

3 faecal pellets from the MD4 mice were collected freshly into 1.5 ml Biopure tube and homogenized. Large debris and cells were removed by centrifugation at 400 g for 5 minutes at 4° C. Supernatant was filtered through 40 μm cell strainer and centrifuged at 400 g for 5 minutes. This step was repeated until no visible pellet was observed. Supernatant was then centrifuged at 8000 g for 10 minutes to pellet bacteria. The pellet was stained with anti-IgA-PE (eBioscience™, 12-4204-82, 1:200) followed by anti-PE microbeads (Miltenyi, 1:200) and sorted on LS columns (Miltenyi) using MACS. Positive fraction was subsequently stained with anti-IgA-AF647 (SouthernBiotech, 1040-31, 1:100). 10⁶ IgA+ and 10⁶ IgA-events were sorted by FACS as PE+AF647+ or PE−AF647-, respectively, centrifuged at 8000 g for 10 minutes and stored at −80° C. until further processed.

16S rRNA Gene Library Preparation and Sequencing

V1-V2 hypervariable regions of 16S rRNA gene were amplified using modified 27F and 338R universal primers. The nucleotide sequences were as following: 27F-5′: AATGATACGGCGACCACCGAGATCTACACTATGGTAATTCCAGMGTTYGATY MTGGCTCAG-3′ and 338R-5′-CAAGCAGAAGACGGCATACGAGATNNNNNNNNNNNNAGTCAGTCAGAAGCTG CCTCCCGTAGGAGT-3′, bold: Illumina adaptor sequences, italic: linkers, NNNNNNNNNNNN sample-specific MID tag barcodes. PCR reactions were performed in duplicates in a volume of 20 μl each using AccuPrime Taq DNA polymerase high fidelity kit (Invitrogen), 4 μl of template DNA and 0.44 μl each primer (stock at 10 μM). PCR programme was as follows: 3 minutes 94° C. (initial denaturation), followed by 30 cycles of: 30 sec 94° C. (denaturation), 30 sec 56° C. (annealing), 1 min 30 sec 72° C. (extension) and 5 min 72° C. (final extension). Duplicates were pooled and amplicon quantity and size determined with the LabChip GX (Perkin Elmer). PCR products were pooled in equimolar amounts and purified using Agencourt AMPure XP magnetic beads (Beckman Coulter). Sequencing was performed on an Illumina MiSeq platform with MiSeq reagent kit V2-500 (pair-end, 2×250).

Shotgun Metagenomics Library Preparation and Sequencing

Bacterial genomic DNA was processes with the TruSeq DNA PCR-Free Low Throughput Library Prep Kit (cat nr 20015962, Illumina). Initial DNA input was 0.5 μg per sample. Shearing was performed using M220 Covaris according to manufacturer's recommendations for 550 bp inserts, except for time of shearing, which was set to 30 seconds. Sheared DNA was further processed using according to manufacturer's recommendations for 550 bp inserts. Library sequencing was performed on an Illumina NovaSeq platform using 2×250 bp chemistry (SP kit).

Shotgun Metagenomics Data Analysis

Shotgun metagenomics data were pre-processed using Sunbeam pipeline for adapter trimming, quality control and mouse genome decontamination (GRCm38 from Genome Reference Consortium) with default parameters. Taxonomic composition and functional

profiling were performed using MetaPhlAn3 and HUMAnN3 pipelines, respectively, with ChocoPhlAn v30 (201901) and the full UniRef90 databases (retrieved Oct. 1, 2020. Gene differential abundance analysis between WT and MD4 tg mice was performed using limma parametric empirical Bayes (eBayes) testing with Imfit function of limma R package (version 3.42.2) on log-transformed data. Differential abundance testing was performed using a Zero-inflated Gaussian mixture model (fitZig function) in metagenomeseq R package (version 1.28.2) and p-values adjusted using Benjamini Hochberg method.

16S rRNA Gene Sequencing Data Analysis

All 16S rRNA gene sequencing analyses were performed in R statistical software. Raw fastq files were demultiplexed and processed using the custom microbiome-dada2 pipeline (https://github.com/respiratory-immunology-lab/microbiome-dada2) with default parameters.

Taxonomic classification and exact sequence matching were performed using SILVA database v123.

Amplicon Sequence Variants (ASVs) filtering, normalisation, ordination, and diversity analyses were performed using phyloseq R package and visualised using ggplot2 R package. Only samples with >1000 Amplicon Sequence Variants (ASVs) were considered for downstream analyses. Unclassified ASVs at Phylum level were removed and filtered based on prevalence (25% of total samples) and counts (100 reads minimum). ASVs count table then was normalised using Total Sum Scaling (TSS). Principal Coordinate Analyses (PCoA) and Analysis of Similarities (ANOSIM) were performed using Bray-Curtis distance matrix calculated using vegan R package. Differential ASV abundance testing was performed using a Zero-inflated Gaussian mixture model (fitZig function) in metagenomeseq R package. For both ANOSIM and differential abundance testing, a model including the genotype (or recolonization genotype) as an explanatory variable and controlling for experiment variation was implemented. Correlation network was inferred using CClasso method (http://github.com/huayingfang/CCLasso/blob/master/R/cclasso.R). Correlation weights with a p-value<0.05 and a correlation coefficient>0.2 were considered significant. IgA binding scores were calculated as following: for each ASV of each sample an IgA+ and IgA fractions relative abundance ratio was calculated and followed by a mean relative abundance ratio if consistent (minimum 1 and higher than 10) in 2 of 3 samples. Network was constructed using igraph R package (version 1.2.5). The formula used to calculate IgA binding index is as follows: relative abundance (IgA+)/relative abundance (IgA−)≥10.

Non-Targeted Metabolite Profiling and Data Analysis

Metabolite profiling was performed by Metabolomic Discoveries GmbH (14476 Potsdam, Germany). Briefly, plasma metabolites from WT and MD4 mice were extracted with 90% methanol/10% water while shaking at 37° C. at 1000 rpm. High resolution mass spectrometry was combined with modified hydrophilic interaction chromatography and the samples were randomised on an Agilent 1290 UHPLC system (Agilent, Santa Clara, United States) equipped with a ZIC-HILIC column (10 cm per 2.1 mm, 3 μm, Sequant, Merck), coupled to 6540 QTOF/MS detector (Agilent, Santa Clara, United States). The detection range was 50-1700 m/z (positive and negative ESI mode). Data were analysed with XCMS, IPO-R package (data conversion, chromatogram peaks extraction), Mzmatch.R (peak filtering and annotation), IDEOM (noise and artefact elimination, putative peak annotation by exact mass±10 ppm). Data was normalized applying Normalization using Optimal selection of Multiple Internal Standards and Cross-Contribution compensating Multiple standard Normalization. Differential abundance analysis of metabolites between WT and MD4 mice was performed on log-transformed data using Imfit function of limma parametric empirical Bayes (eBayes) testing with Imfit function of limma R package (version 3.42.2) and p-values adjusted using Benjamini Hochberg method.

Isolation of Lung Cells and Airway Epithelial Cells

Mice were euthanized by CO2 inhalation, instilled with a 1.5 ml dispase II (Sigma-Aldrich, St. Louis, Mo.) intratracheally, followed by intratracheal injection of 0.5 ml 1% low melting point agarose (Sigma-Aldrich, St. Louis, Mo.). Lungs were then covered with ice for 3 minutes, removed and placed in a 15 ml falcon tube with 2 ml of dispase II, and incubated for 45 minutes with gentle agitation. This was followed by mechanical disruption of the lung lobes using forceps in DMEM supplemented with DNAse I (Sigma-Aldrich, St. Louis, Mo. 1U/ml), filtration through 70 and 40 um cell strainers (FalconR, Corning) and lysis of erythrocytes using red blood cell lysing buffer (BD Biosciences). Lung cells were plated in a flat bottom 24-well or 96-well plates (Costar) coated with fibronectin (Sigma-Aldrich, St. Louis, Mo.; 10 ug/ml) at the density of 1 min or 0.2 min cells/well, respectively.

Targeted Metabolomics for PCS

25 μL of plasma samples were extracted with 100 μL of chilled methanol containing internal standard (PCS-d4 at 500 ng/ml plasma concentration), shaken on ice for 30 min and centrifuged at 4° C. for 10 min. 100 μL of supernatant was diluted with 100 μL of 0.1% FA in water. Frozen fecal samples were weighted (3-5 mg) and extracted with 20 μL/mg of 80% chilled methanol containing internal standard PCS d4, vortexed at 4° C. for 15 min, shaken at 25° C. 60 min and centrifuged at 4° C. at 14800 g for 30 min. Supernatant was collected and diluted 2.5× with 0.1% FA. PCS-d4 concentration is 50 ng/ml in the samples which is equal to 2.5 ng/1 mg faeces). 50 μL of BAL fluid were extracted with 200 μL of cold methanol, mixed on ice for 30 min and centrifuged at 4° C. at 14800 g for 10 min. 200 μL of the supernatant was transferred to new Eppendorf tubes and evaporated under nitrogen stream for 60 min at 20° C. Samples are resolubilized in 100 μL 0.1% FA in water, mixed for 15 min at 25° C., sonicated with ice for 15 min, centrifuged at 4° C. and transferred to vials. Samples were analyzed on the same day as prepared injecting 6 μL and using the following LCMS acquisition method: LCMS data was acquired on Q-Exactive mass spectrometer coupled with Dionex Ultimate 3000 RSLC separation system (Thermo ScientificAscentis Express C8 (100×2.1 mm, 2.7 μM, Supelco) column protected with a guard column (C8, 2×2 mm, Phenomenex) was used for separation. Buffer A was 0.1% formic acid in water and buffer B was 0.1% formic acid in acetonitrile. Gradient elution was achieved starting at 10% B concentration and increased to 95% B in 3.5 min, kept at 95% B until 4.5 min, reduced to 10% B at 5 min and equilibrated at that ratio until 7 min. Autosampler temperature was kept at 4° C. and column oven at 40° C. HESI source spray voltage was set to 4 kV, capillary temperature 300° C., auxiliary gas temperature 120° C., sheath gas flow rate to 50, auxiliary gas to 20, sweep gas to 2 arbitrary units and S-lens RF level 50. Mass spectrometer operated in PRM acquisition mode in negative ion polarity using inclusion list for PCS and PCS-d4 m/z (m/z 187.0071 and 191.0321, respectively) with specified HCD collision energy NE=50 and retention time between 2-3.5 min. Other PRM parameters were as follows: 1 microscan, 17.5 k resolution, AGC target 2e5, maximum IT 100 ms, isolation window 2 m/z, loop count 4, MSX count 1. Peak integration and quantitation were performed using Tracefinder 4.1 application (Thermo Scientific).

Isolation of Lung Cells Enriched for an Airway Epithelial Cell Fraction

Mice were euthanized by CO2 inhalation, instilled with a 1.5 ml dispase II (Sigma-Aldrich) intratracheally, followed by intratracheal injection of 0.5 ml 1% low melting point agarose (Sigma-Aldrich). Lungs were then covered with ice for 3 min, removed and placed in a 15 ml falcon tube with 2 ml of dispase II, and incubated for 45 min with gentle agitation. This was followed by mechanical disruption of the lung lobes using forceps in DMEM supplemented with DNAse I (Sigma-Aldrich, 1U/ml), filtration through 70- and 40-μM cell strainers (Falcon®, Corning) and lysis of erythrocytes using red blood cell lysing buffer (BD Biosciences). Lung cells were plated in a flat-bottom 24-well or 96-well plates (Costar) coated with fibronectin (Sigma-Aldrich, 10 μg/ml) at the density of 1 mln or 0.2 mln cells/well, respectively.

In Vitro Stimulation of Lung Cells

Cells were stimulated with HDM (Greer; 100 μg/ml), LPS (Sigma Aldrich, 10 μg/ml), PCS (Alsachim, Illkirch-Graffenstaden, France; 100 μg/ml), Epidermal Growth Factor (Thermo Fisher Scientific; 100 ng/ml); Amphiregulin (In Vitro Technologies; 500 ng/ml) or Gefitinib (Sigma Aldrich, 0.16 μM) for 24 hours at 37° C. 5% CO2, after which the supernatant was collected and stored at −20° C. until further use.

### Example 2: Mice with a Restricted Antibody Repertoire do not Develop Allergic Airway Disease

To model a lack of antibody diversity, a transgenic mouse strain with an antibody repertoire fixed to a single model antigen, hen egg lysozyme (HEL) (hereafter referred to as MD4 mice) was used. Given the positive correlation between diversification of the antibody repertoire and a diverse microbiota—a characteristic associated with health benefits—the present inventors hypothesized that the MD4 mice would have a reduced microbial diversity and consequently, an increased susceptibility to inflammation, such as allergic airway inflammation, a mouse model of asthma.

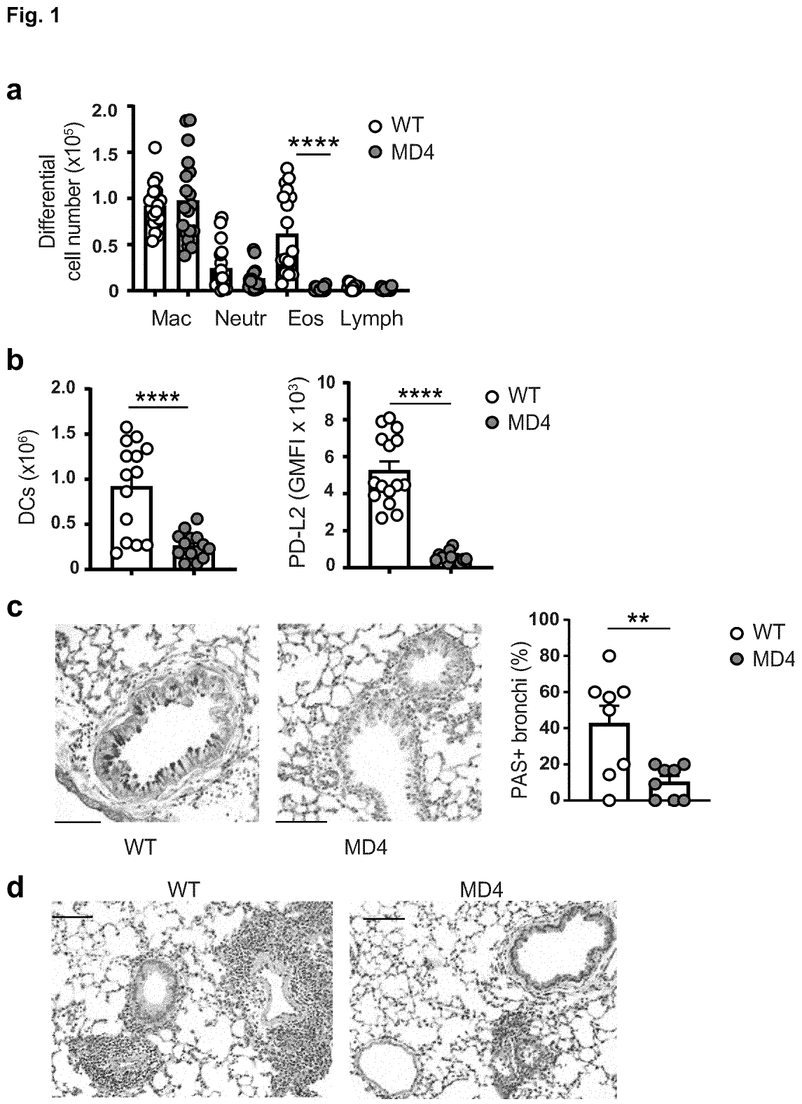

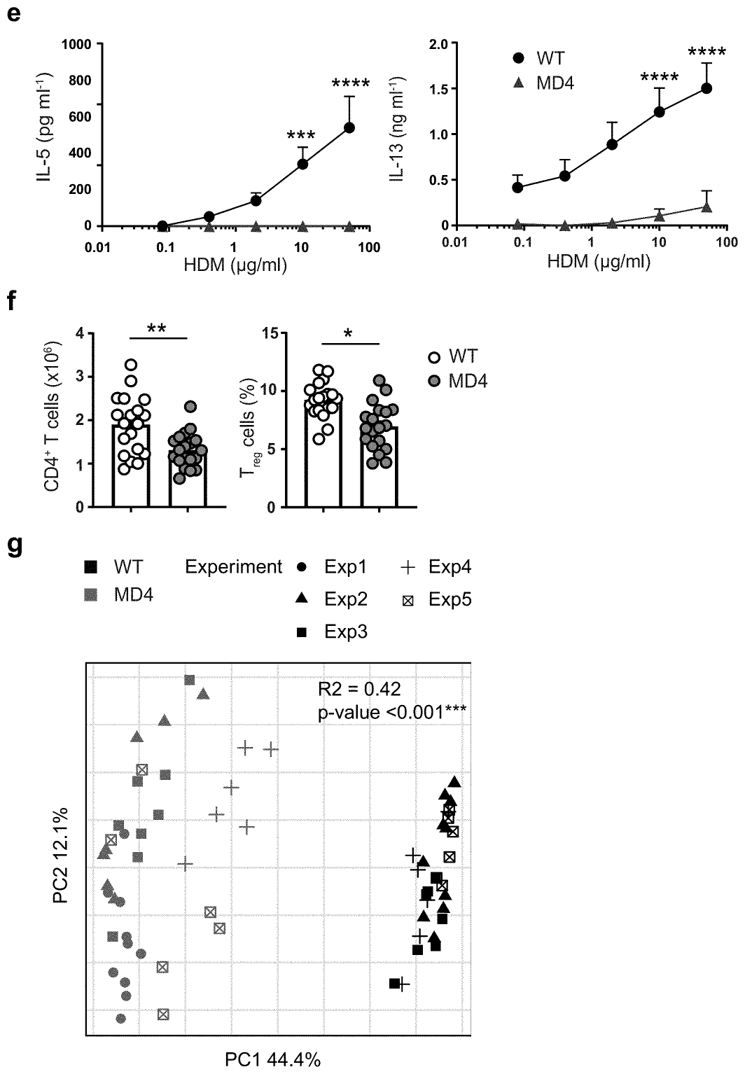

On the contrary, intranasal exposure of MD4 mice to house dust mite (HDM) extract (FIG. 1) unexpectedly led to an almost complete absence of the allergic airway disease seen in wild-type controls, including eosinophilia (FIG. 1a), recruitment and activation of pulmonary DCs (FIG. 1b), mucous production (FIG. 1c), goblet cell hyperplasia and lung pathology (data not shown), peribronchial and perivascular inflammatory cell infiltrates (FIG. 1d) and the production of the Th2-associated cytokines interleukin 5 (IL-5) and IL-13 (FIG. 1e).

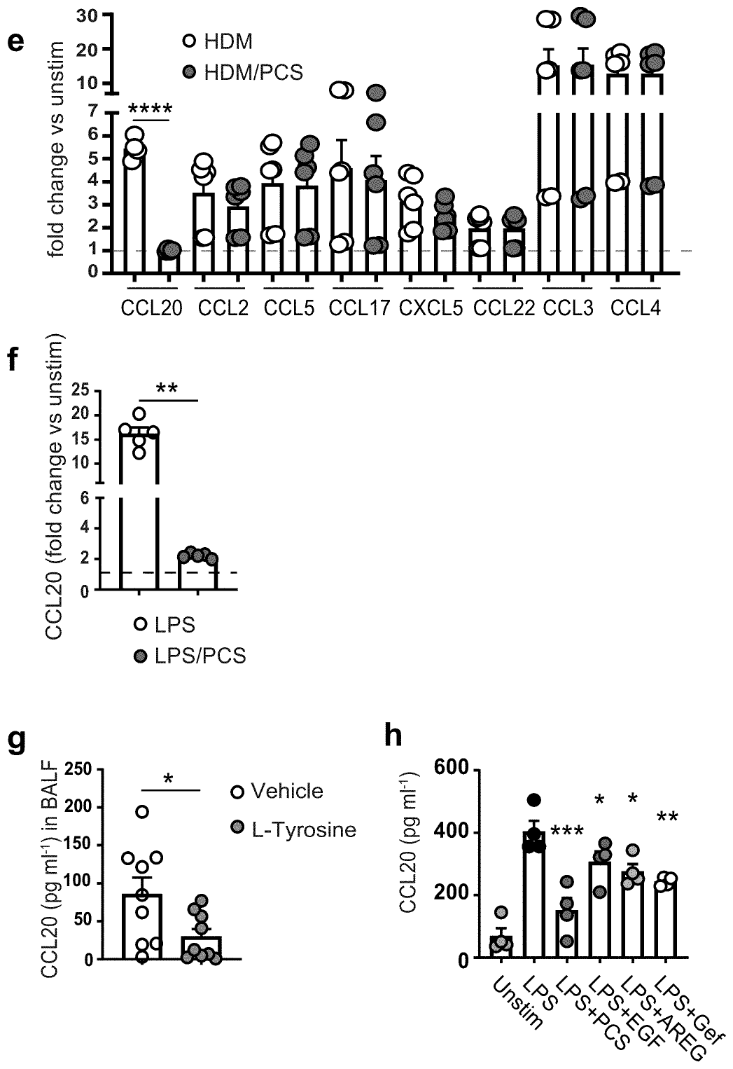

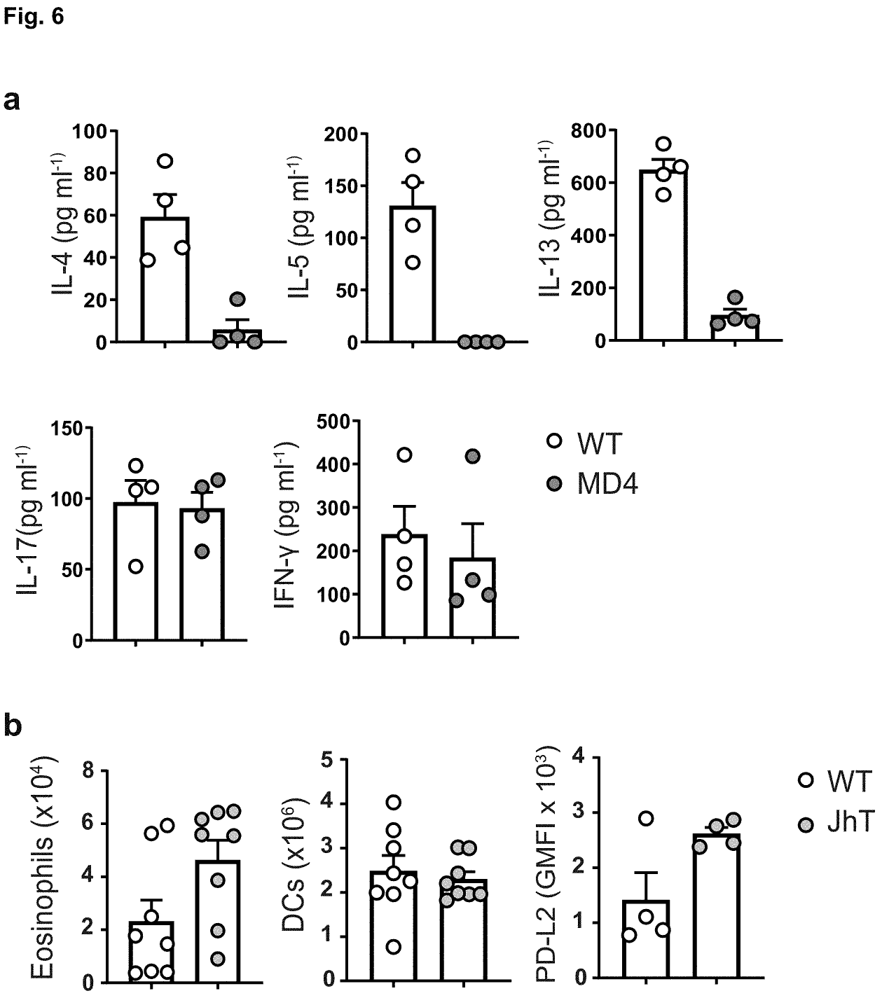

Because the cellular composition of mediastinal lymph nodes markedly differs between WT and MD4 mice exposed to HDM (as a consequence of the lack of B cell proliferation in the latter), CD4+ T cells were sorted from the lymph nodes of both groups and co-cultured with dendritic cells in the presence of HDM. Consistent with FIG. 1e, type 2 cytokines were not detected in culture supernatants of MD4 T cells (FIG. 6a). Recruitment of CD4+ T cells was only moderately decreased with a slight reduction in the proportion of FoxP3+ regulatory T cells (Tregs) (FIG. 1f). In contrast, B cell deficient mice (JhT) mounted an allergic response similar to that seen in wild-type mice (FIG. 6b), indicating that the protection of the MD4 strain was not due to the absence of antigen-specific B cells.

These data demonstrate mice with a restricted antibody repertoire do not develop allergic airway disease.

### Example 3: Microbiota Confers Protection Against Allergic Airway

Inflammation and Transfer of Microbiota Ameliorates Allergic Airway Inflammation

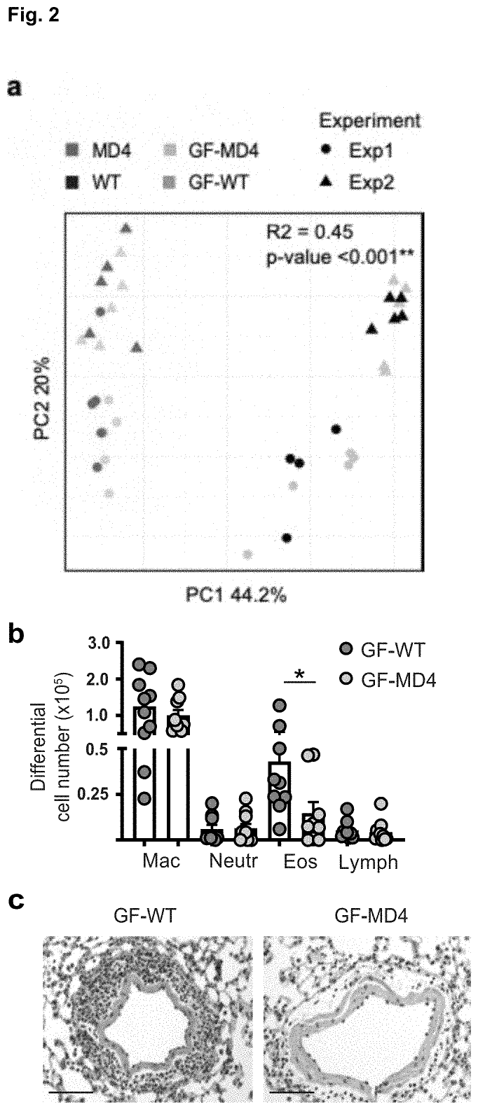

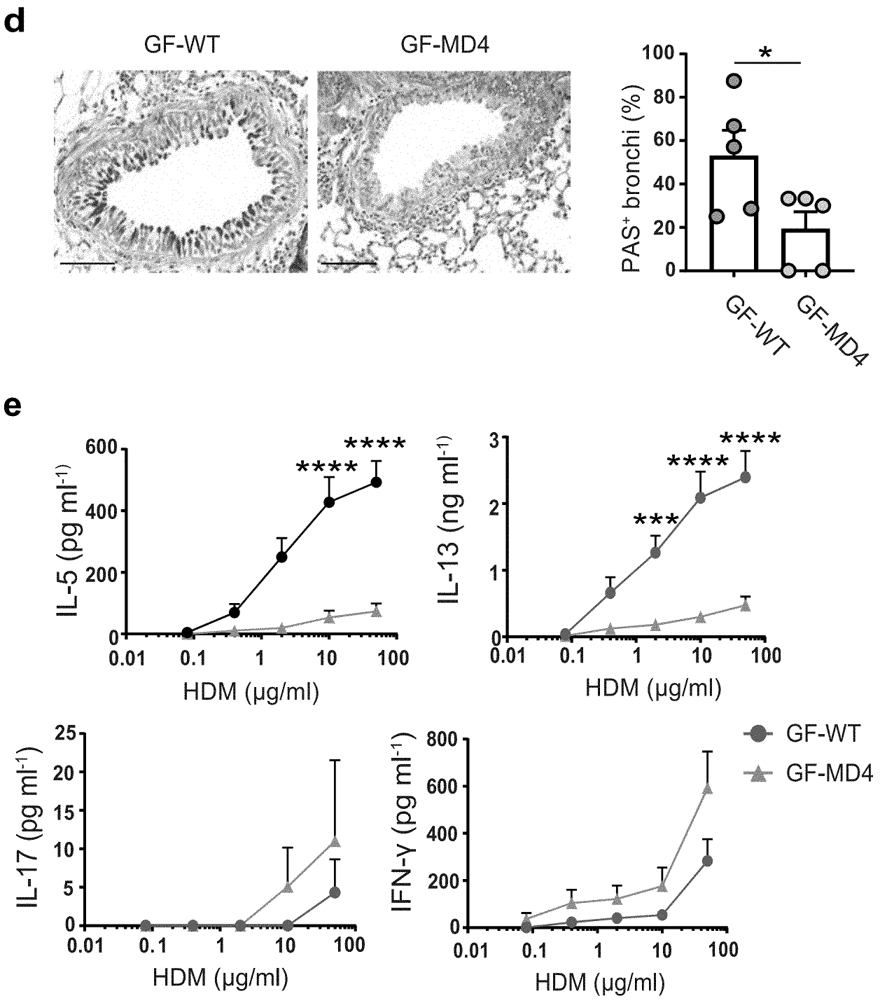

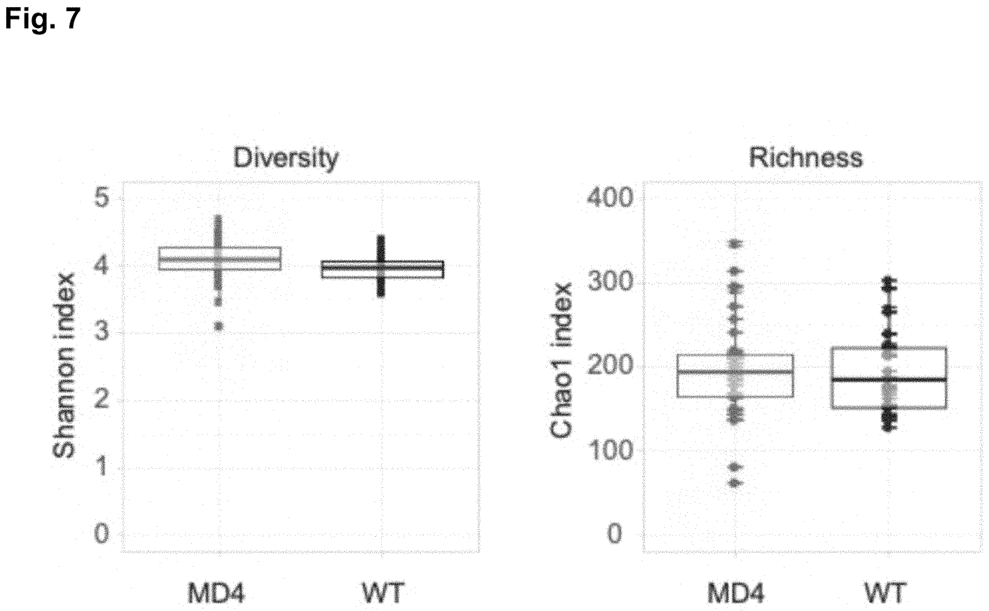

MD4 mice did not have major alterations in microbiota diversity (FIG. 7), but had substantial differences in the composition of the microbiota (FIG. 1g). To evaluate whether the microbiota contributed to the observed protection, germ-free (GF) mice were co-housed with either wild-type (WT) or MD4 mice for 6 weeks, after which they were exposed to HDM (FIG. 2). Sequencing of 16S rRNA gene amplicons from faecal DNA confirmed acquisition of the MD4 microbiota by co-housed germ-free mice (GF-MD4) (ANOSIM, F=19.69, R2 0.45, p-value<0.001) (FIG. 2a).

Transfer of the MD4 microbiota ameliorated allergic responses, including airway eosinophilia (FIG. 2b), lung pathology (FIG. 2c), mucus production (FIG. 2d), secretion of Th2-associated cytokines (FIG. 2e) and production of HDM-specific antibodies (FIG. 2f). However, there were no alterations in the recruitment of CD4+ T cells or the proportion of FoxP3+67 Treg cells (FIG. 2g).

These data demonstrate microbiota of the MD4 mice confers protection against HDM-induced allergic airway inflammation. In particular, these data demonstrate the microbiota of the MD4 mice ameliorated eosinophilia, production of HDM-specific antibodies, lung pathology, goblet cell hyperplasia, mucus production, and secretion of Th2-associated cytokines.

These data also demonstrate that transfer of the MD4 microbiota ameliorated allergic responses. In particular, these data demonstrate that the transfer of the microbiota of the MD4 mice ameliorated eosinophilia, production of HDM-specific antibodies, lung pathology, goblet cell hyperplasia, and secretion of Th2-associated cytokines.

### Example 4: Mice that do not Develop Allergic Airway Disease have Alterations in the Microbiome and the Metabolome, Including Elevated Levels of p-Cresol Sulfate

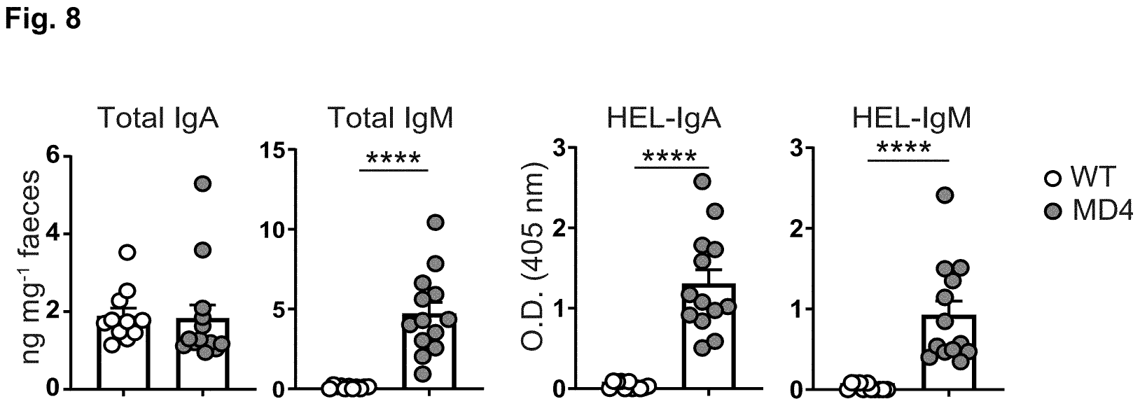

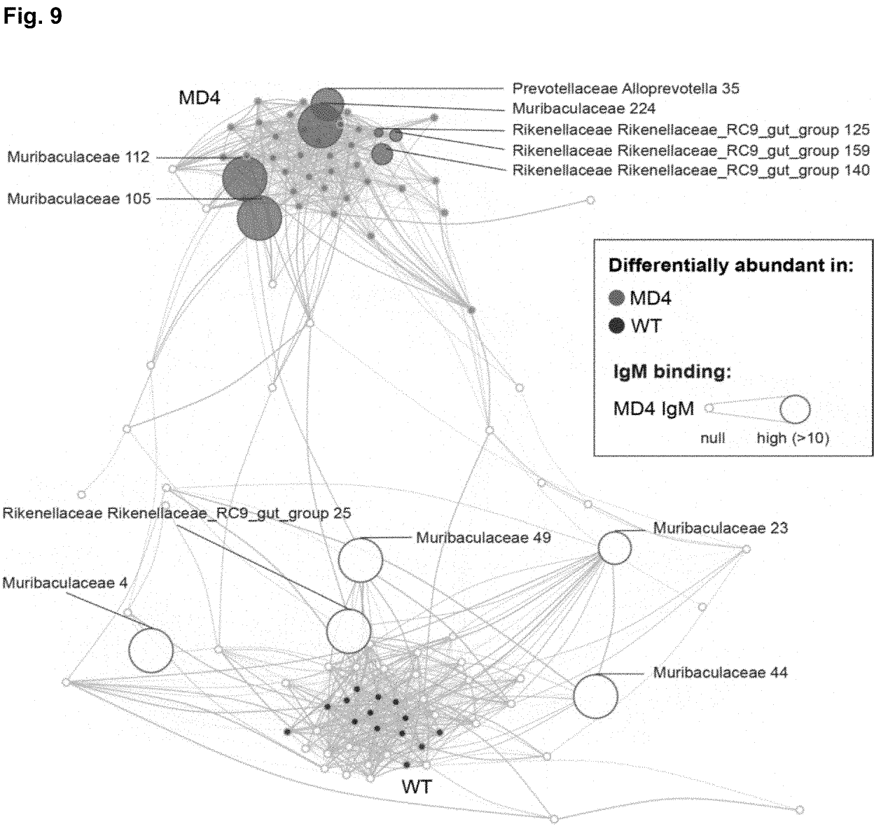

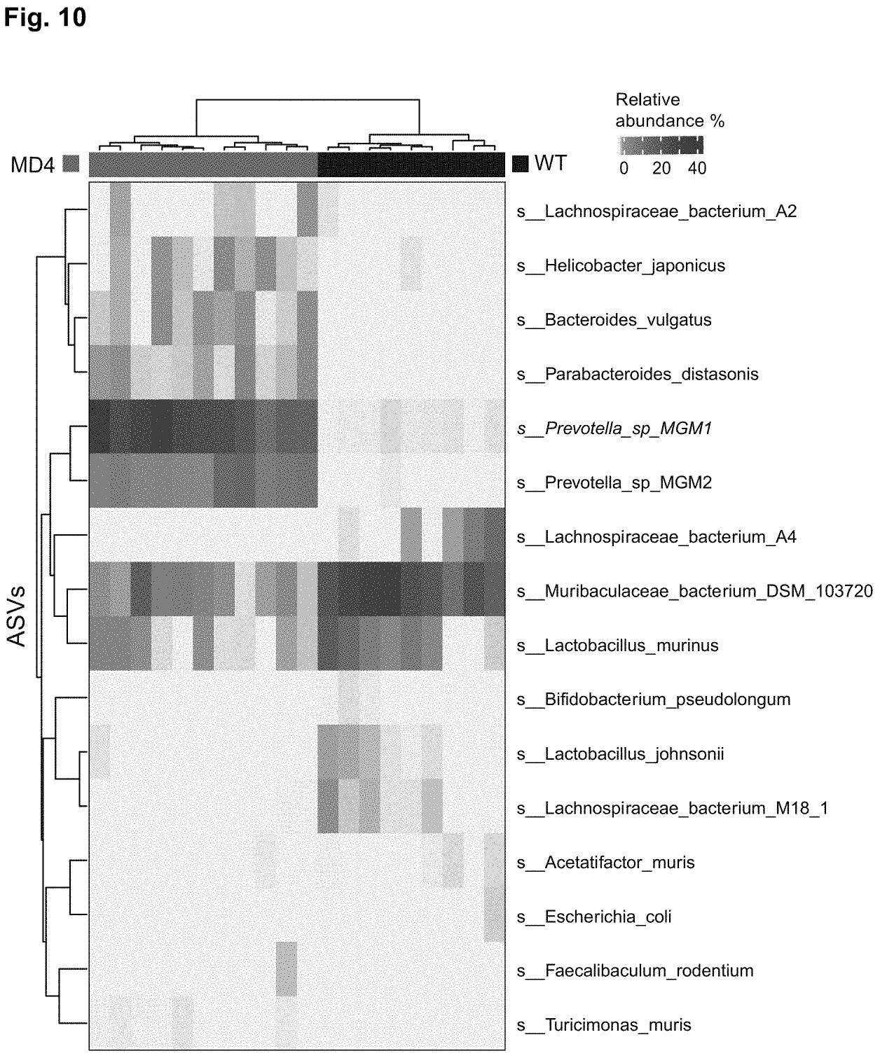

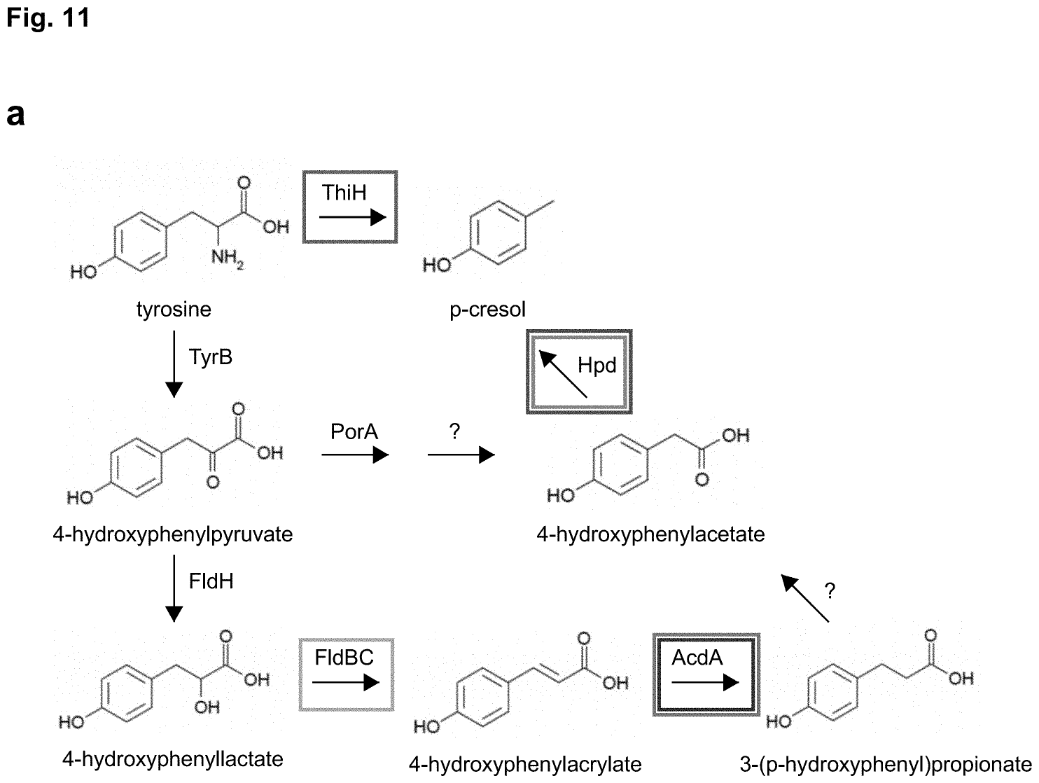

Next, the microbiota composition of the MD4 mice was analysed in depth. 16S rRNA gene sequencing revealed 41 bacterial taxa with increased abundance, and 14 with decreased abundance in this mouse strain (FIG. 3a); 7 out of these 41 taxa, and 0 out of the 14, were coated with secretory IgA (FIG. 3b, blue nodes), which was found in high abundance in the MD4 faeces and displayed specificity to HEL (FIG. 8). MD4 IgA binding showed no overlap with that of WT mice (FIG. 3b, black nodes). IgM, which was highly abundant in MD4 faeces (FIG. 8), showed a similar binding pattern, coating 5 out of the 7 IgA-coated taxa (FIG. 9). The exact nature of the antibody-microbe interactions is not clear; they may be facilitated by the cross-reactivity of antigen-binding (Fab) regions of the anti-HEL antibodies or by non-Fab dependent affinities (e.g. that of a secretory component). Although the mechanisms remain to be fully elucidated, it is clear that restricting the antibody repertoire to HEL alters the microbial community. Because gut microbes can have distal immunomodulatory effects through the release of metabolites into the circulation, the metabolome of MD4 mice was assessed. Untargeted plasma metabolomic profiling was performed, and identified p-cresol sulfate (PCS) as the metabolite with the strongest enrichment (Limma, Log FC=3.1, adj.p.val=1.42E-06) in the MD4 mice (FIG. 3c). PCS is a sulfation product of p-cresol (FIG. 3d), the intestinally generated microbial-derived product of L-tyrosine metabolism. P-cresol sulfation takes place in the mucosa of the colon, and in the liver. In line with a microbial origin of PCS, germ-free mice co-housed with MD4 mice also showed increased concentrations of PCS (FIG. 3e), suggesting the MD4 microbiota had a superior capacity to utilize L-tyrosine from the diet. Indeed, shotgun metagenomics analyses of fecal samples revealed ThiH genes encoding for enzymes involved in the direct conversion of L-tyrosine to p-cresol, were more abundant in the MD4 microbiota (log FC 3.9, adj.p.val=5.54E-04 and log FC 3.2, adj.p.val=5.62E-07) (FIG. 3f). One of these genes mapped to the Prevotella MGM1 species genome, highly abundant in the MD4 mice (FIG. 10), while the other was from an unidentified source. An alternative pathway for production of p-cresol from L-tyrosine involves the bacterial genes TyrB, FldH, PorA, FldBC, AcdA and HpD (FIG. 11a). TyrB, FldH or PorA were not detected, while FldBC and HpD were not differentially abundant between the groups. Sequences mapping to AcdA were found to be differentially abundant in both wild-type and MD4 groups. However, differentially abundant AcdA genes in wild-type samples related to different putative proteins (Acda C-terminal domain) than the ones enriched in in MD4 faeces (FIG. 11b). Given AcdA is involved in multiple pathways, not just those leading to p-cresol, the present inventors conclude ThiH, which metabolizes L-tyrosine to PCS in a single step, is the metabolic enzyme relevant in the model described herein. The faeces of MD4 mice were also found to contain less L-tyrosine, supportive of the conclusion that the microbiota of these mice exhibited enhanced metabolism of this amino acid in the gut (FIG. 3g).

These data demonstrate that mice that do not develop allergic airway disease have alterations in the microbiome and the metabolome, including elevated levels of p-cresol sulfate.

These data also demonstrate cross-reactivity of anti-HEL IgA contributes to changes in the microbiome and the metabolome of the host.

### Example 5: Administration of PCS or L-Tyrosine Protects Against Allergic Airway Inflammation

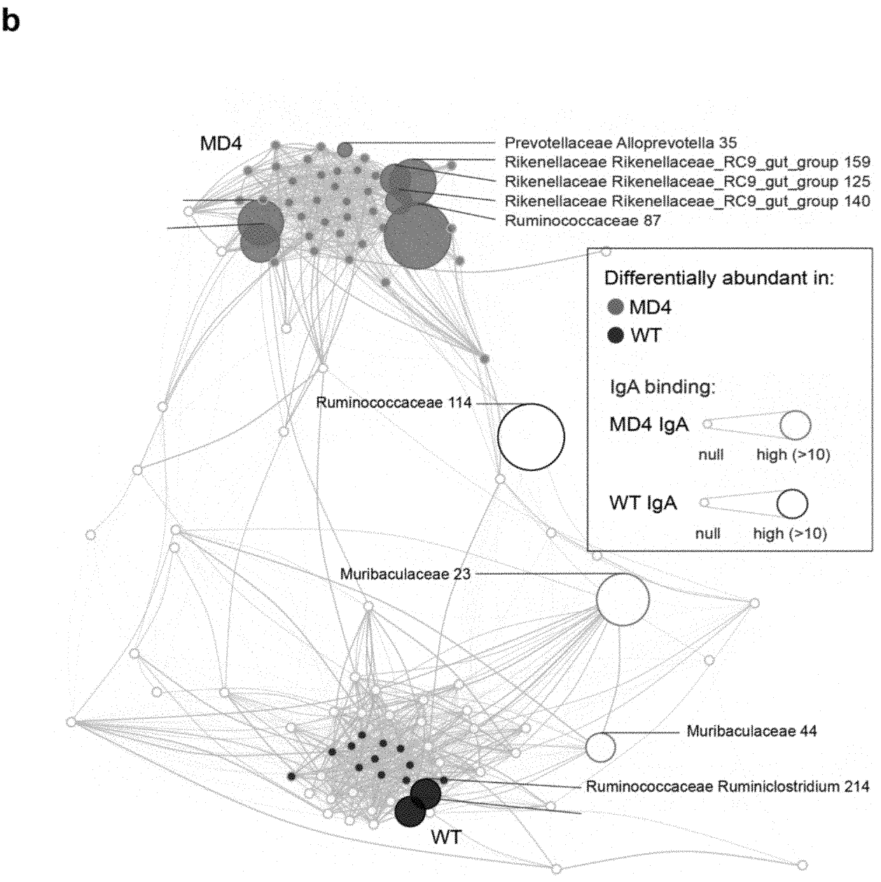

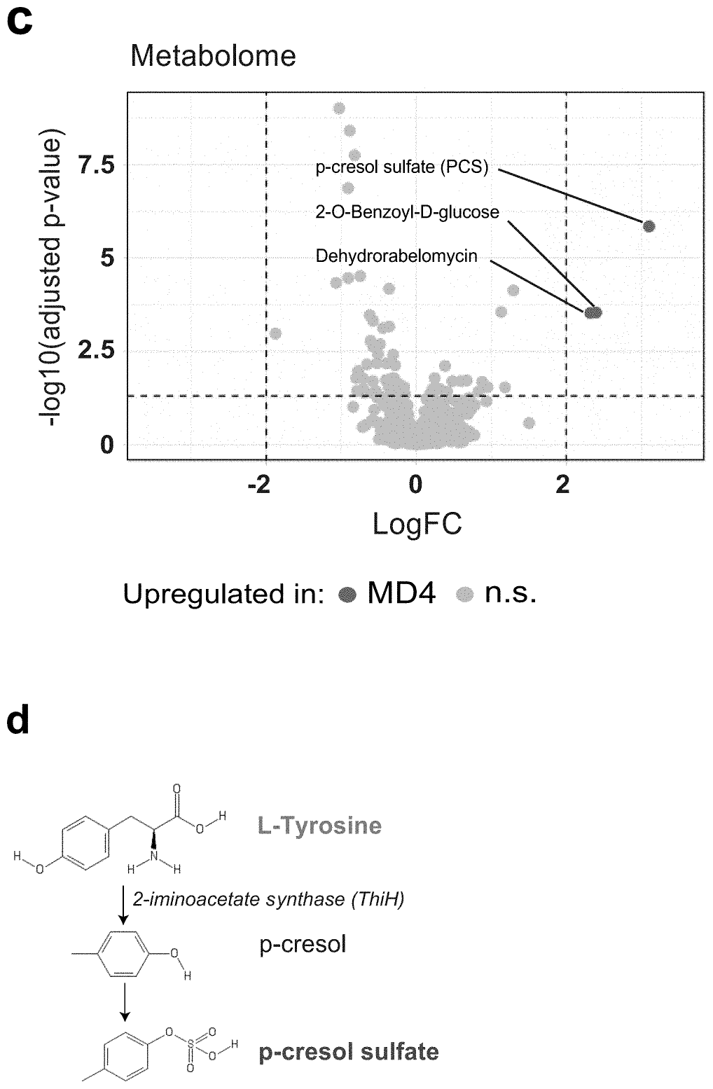

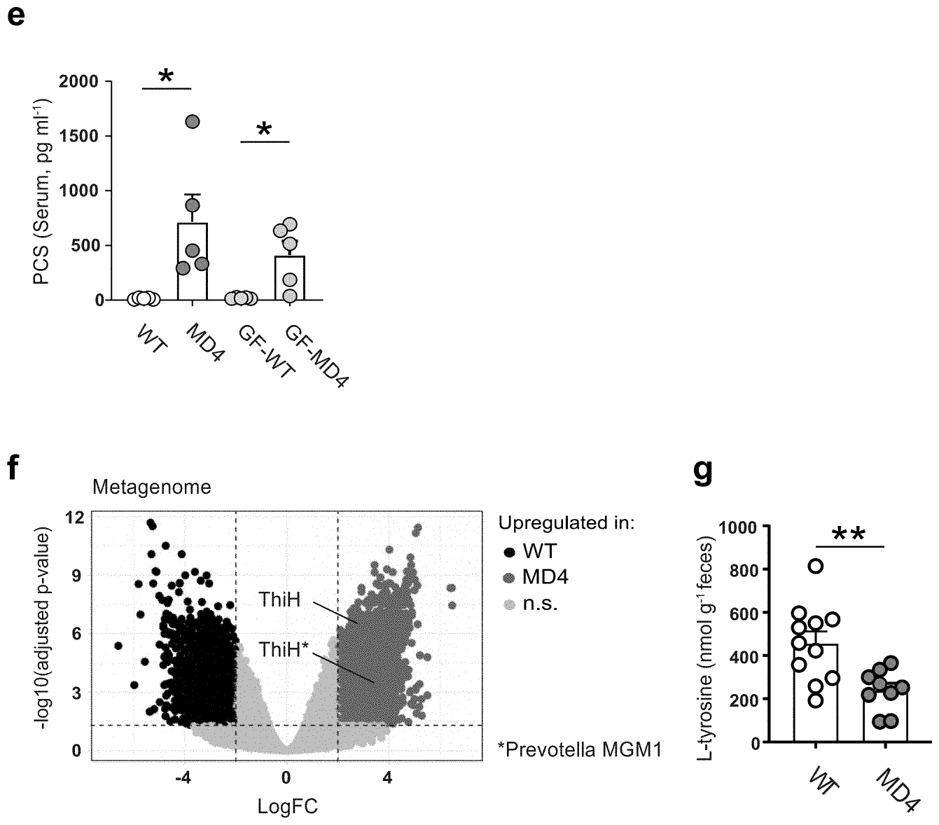

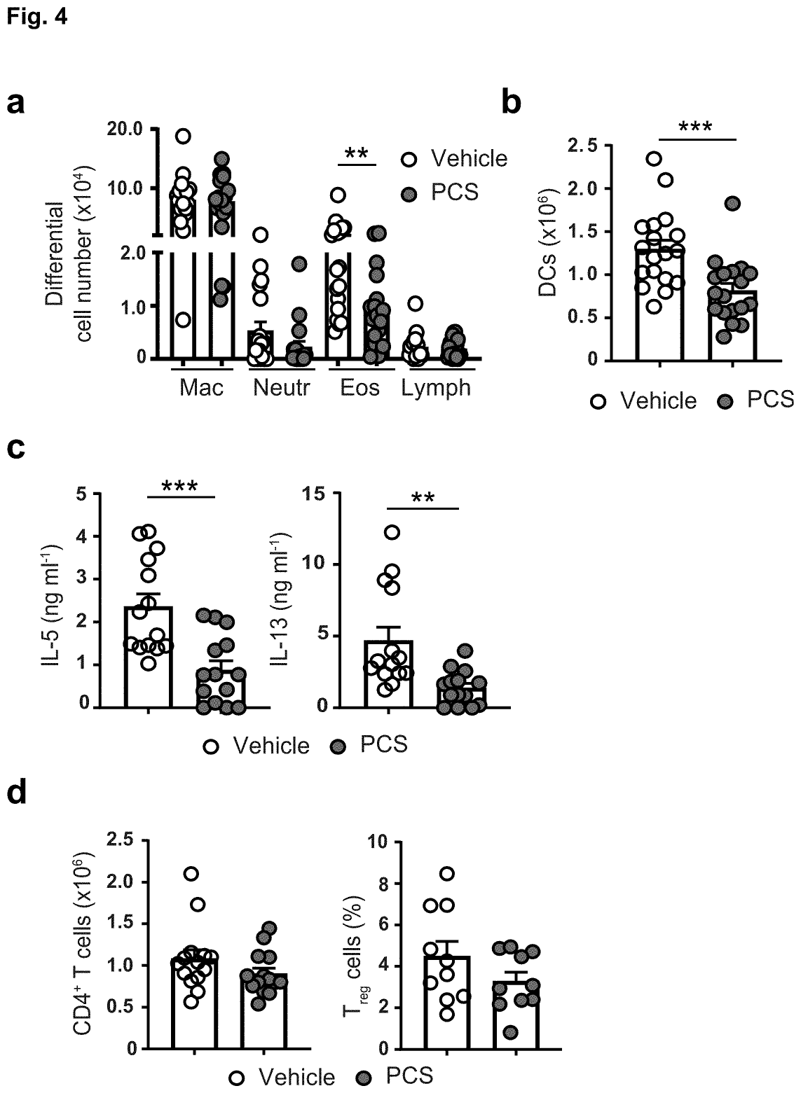

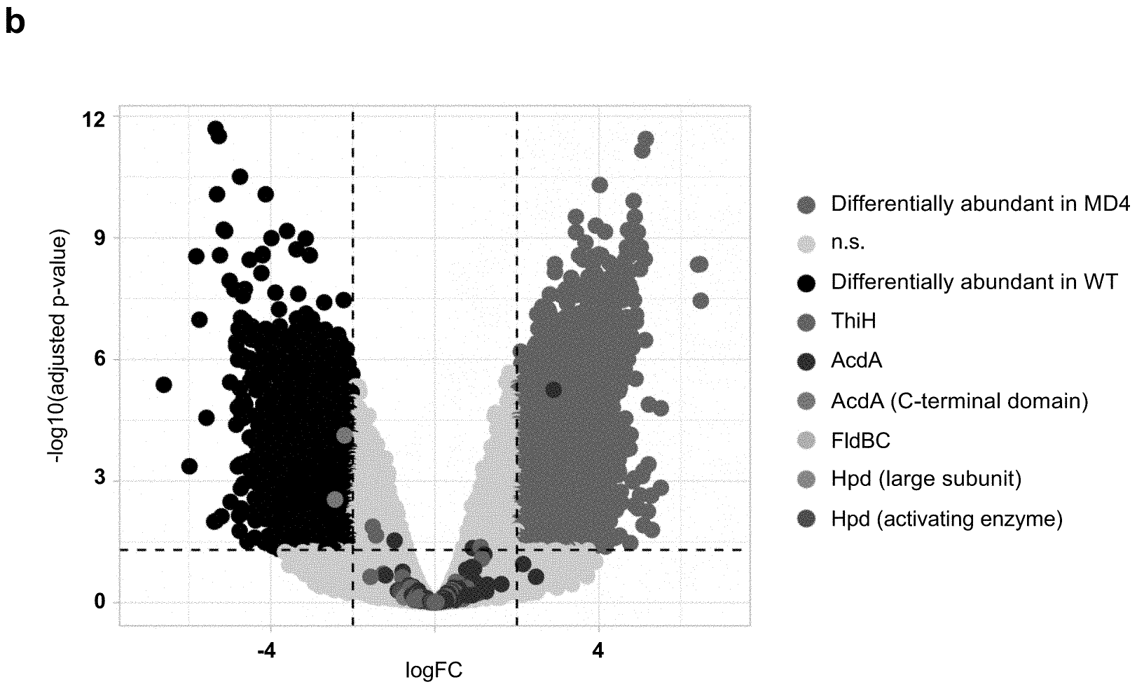

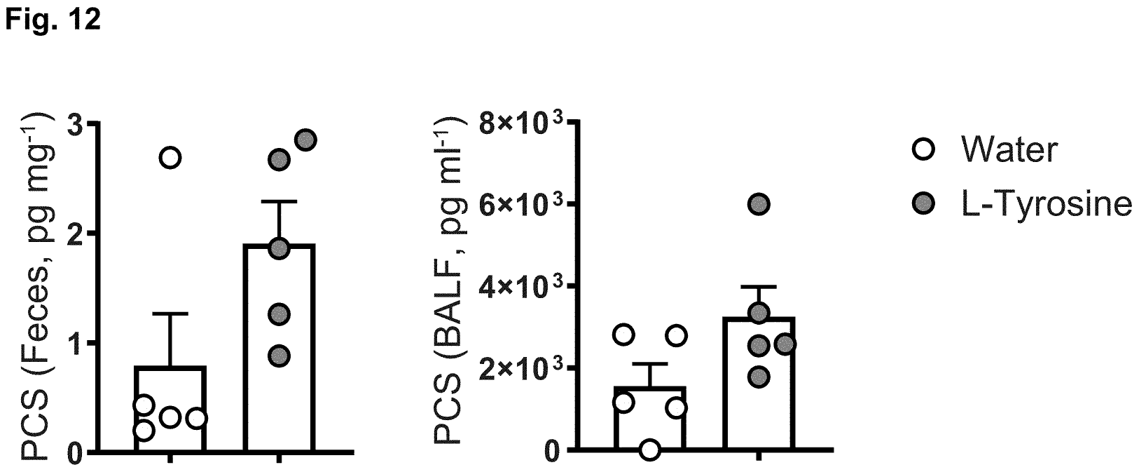

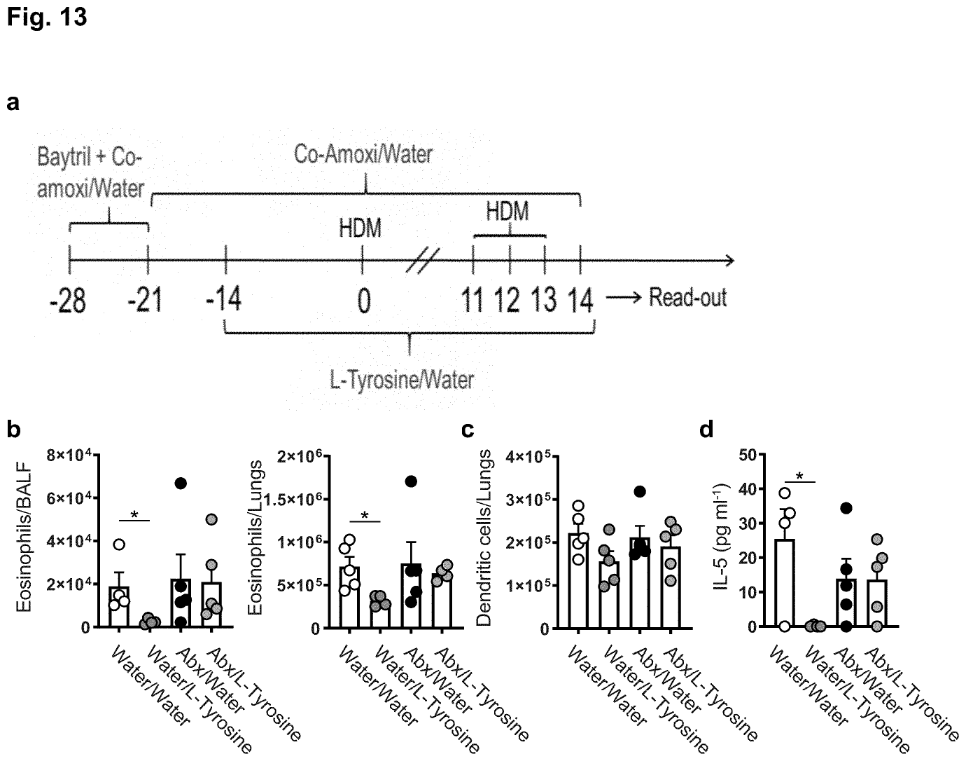

The influence of PCS or L-tyrosine treatment on allergic airway inflammation was investigated. Wild-type C57BL6/J mice received intravenous injection of PCS or saline prior to HDM sensitization and challenge (FIG. 4). This treatment ameliorated the eosinophilia in the BALF (FIG. 4a), decreased infiltration of DCs into the lungs (FIG. 4b) and reduced production of IL-5 and IL-13 by restimulated mediastinal lymph nodes (FIG. 4c). As seen with the MD4 mice, there were no major alterations in the numbers of lung CD4+ T cells or FoxP3+Tregs (FIG. 4d). Oral administration of L-tyrosine (FIG. 4) led to an increase of PCS concentrations in the faeces and airways (FIG. 12) and conferred similar effects: reduced recruitment of eosinophils, neutrophils and DCs, (FIG. 4e,f), and reduced production of IL-13 (FIG. 4g). IL-5 concentrations in culture supernatants of mediastinal LNs showed a trend towards a decrease (FIG. 4g), albeit not reaching statistical significance. Numbers of T helper cells or FoxP3+Treg cells in the lungs were not altered (FIG. 4h,i). Antibiotic treatment (as per FIG. 13a) abrogated the protective effect of L-tyrosine (FIG. 13b-d).

These data demonstrate that administration of PCS or L-tyrosine protects against allergic airway inflammation. In particular, these data demonstrate that administration of PCS ameliorated the eosinophilia in the BALF, reduced recruitment of neutrophils, decreased infiltration of DCs into the lungs and reduced production of IL-5 and IL-13 by restimulated mediastinal lymph nodes.

These data also demonstrate that administration of L-tyrosine reduced eosinophilia, reduced neutrophil and DC recruitment, and reduced production of IL-13. These data also demonstrate that administration of L-tyrosine reduced the production of IL-5 and IL-13 by restimulated mediastinal lymph nodes; reduced eosinophilia (e.g. reduced eosinophilia in bronchoalveolar lavage fluid); and reduced infiltration of pulmonary dendritic cells into the lungs.

### Example 6: L-Tyrosine— PCS Axis Modulates Airway Epithelial Cell—Dendritic Cell Cross-Talk

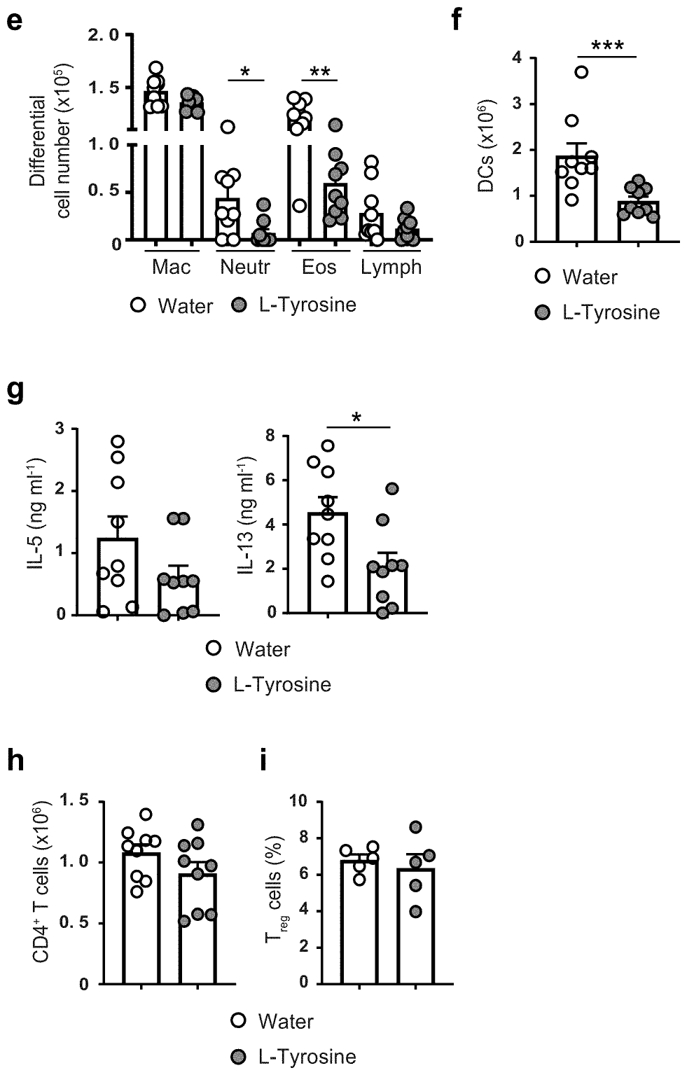

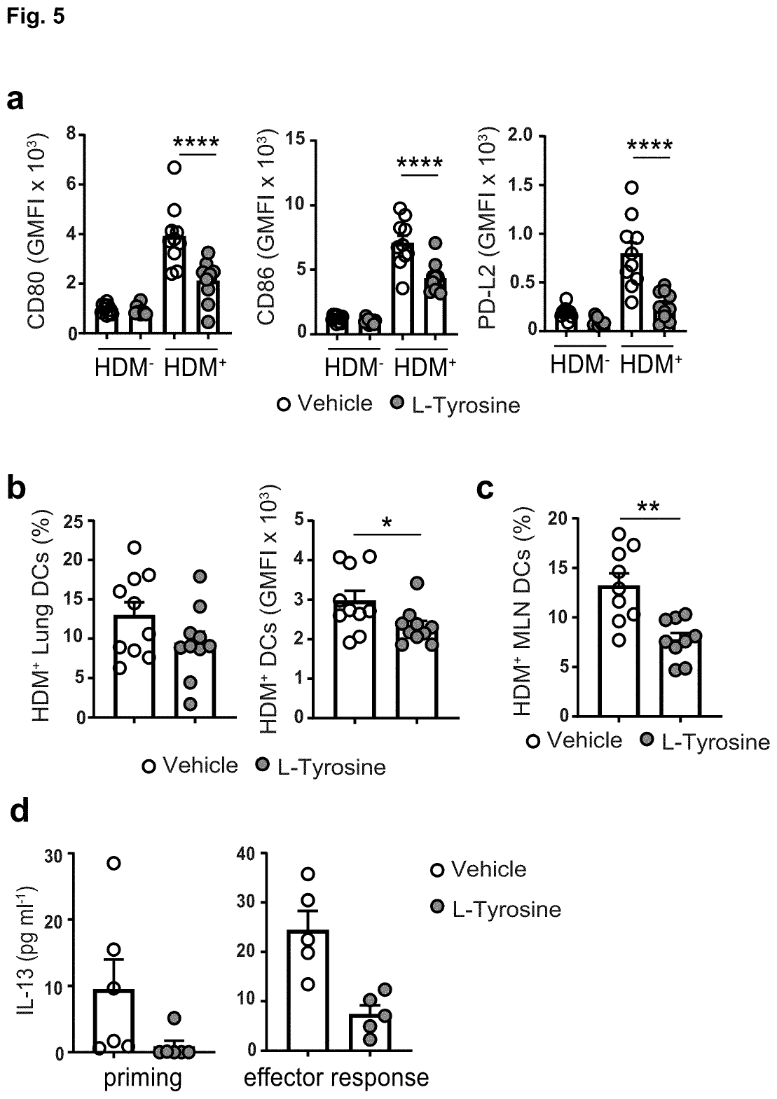

The mechanisms behind the protective effects of L-tyrosine and PCS treatments were investigated. Intranasal administration of fluorescently labelled HDM into L-tyrosine-treated mice (FIG. 5) revealed an impairment in the activation of their lung DCs (FIG. 5a), with only a modest effect on the antigen uptake (FIG. 5b). The migratory capacity of DCs was decreased, as shown by a reduced frequency of HDM+DCs in the draining LNs (FIG. 5c). In addition, their capacity to prime naive CD4+ T cells or restimulate in vivo-primed effector T helper cells into an IL-13-producing subset was impaired (FIG. 5d).

To gain insight into how oral supplementation of L-tyrosine influences DC function, the activity of PCS on chemokine release from HDM-stimulated lung cells isolated using a protocol for epithelial cell enrichment was used. Briefly, lung cells from naïve C56BL6/J mice were isolated using 1% low melting agarose/dispase II solution, plated on fibronectin-coated plates and stimulated in the presence/absence of PCS and HDM.

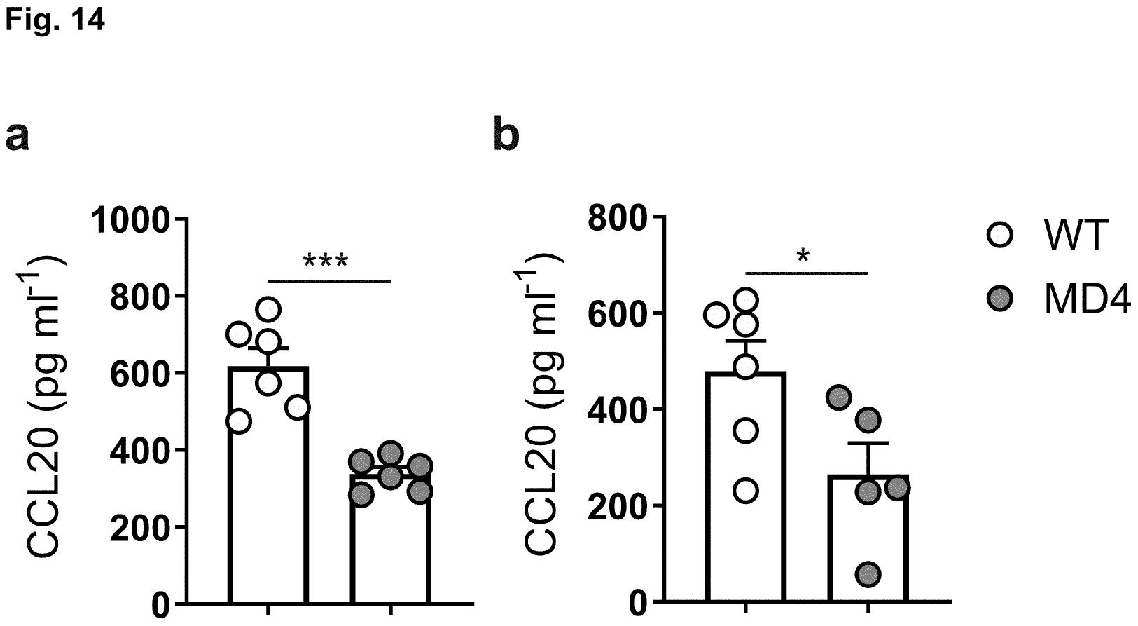

Strikingly, PCS completely abrogated HDM-induced production of an airway epithelial cell-derived DC chemoattractant, CCL20 but did not have an effect on other chemokines (FIG. 5e). A similar observation was noted in lung cells isolated from the MD4 mice (FIG. 14a).

Because HDM induced low levels of CCL20, LPS, a known potent inducer of CCL20, was to further evaluate the efficacy of PCS. LPS induced a 15-fold upregulation of CCL20, which was inhibited by PCS (FIG. 5f). Consistent with these in vitro data, CCL20 concentrations were reduced in the BALF of L-tyrosine-treated wild-type mice (FIG. 5g) and MD4 mice (FIG. 14b) exposed to HDM.

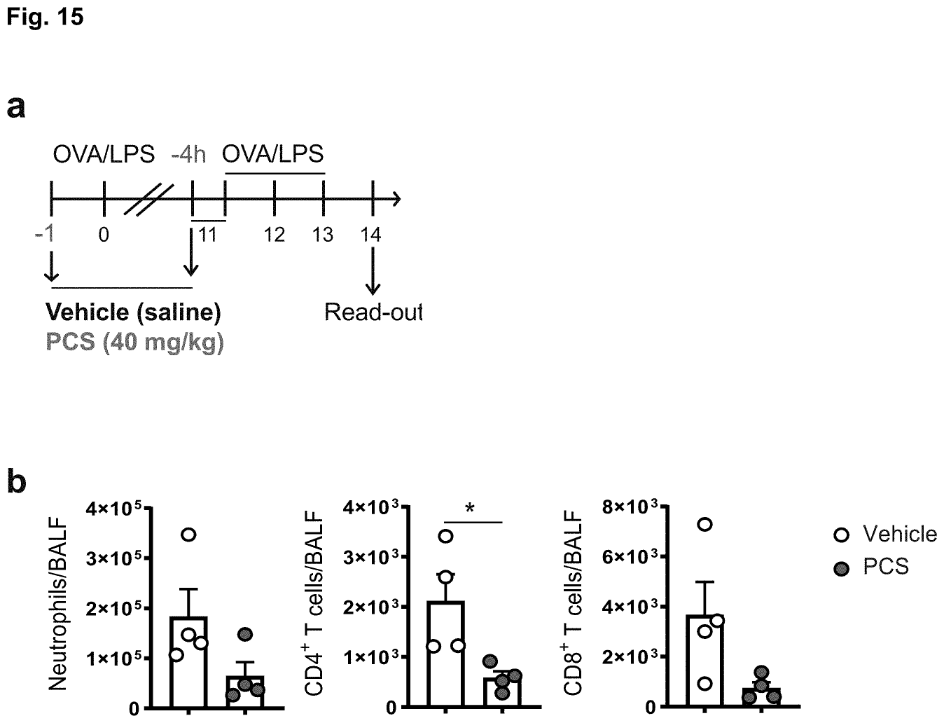

Because CCL20 function is not restricted to type 2 immunity and may influence a broader range of immune responses, the efficacy of PCS in the context of type 1 mediated lung immunopathology was tested. Mice were administered intranasally with ovalbumin/LPS on days 0, 11, 12 and 13, and intravenously with PCS on day −1 and on day 11, 4 hours prior to first ovalbumin/LPS challenge (FIG. 15a). PCS inhibited infiltration of neutrophils, CD4+ and CD8+ T cells to the airways (FIG. 15b).

Molecular docking analysis shows PCS could bind in the interdomain pocket of EGFR, just beneath the EGF binding site. EGFR is required for optimal signal transduction downstream of TLR-4 by facilitating recruitment of Lyn to both receptors. The present inventors proposed that PCS inhibits CCL20 production via uncoupling TLR4 and EGFR cross-talk. To test this, the present inventors stimulated lung cells with LPS in the presence of EGFR ligands (high affinity— EGF and low affinity—amphiregulin) or in the presence of an EGFR inhibitor, gefitinib. As in the case of PCS, all treatments led to a selective reduction in CCL20 production (FIG. 51 and FIG. 10), recapitulating the effect of PCS, albeit with lower efficacy. This data highlighted the importance of an unbound EGFR for TLR-4-mediated production of CCL20 in response to LPS.

These data demonstrate oral administration of L-tyrosine reduced activation of pulmonary dendritic cells; reduced migration of pulmonary dendritic cells into draining lymph nodes, reduced the frequency of HDM+DCs in the draining LNs, and reduced the capacity of DC cells to prime naive CD4+ T cells or restimulate in vivo-primed effector T helper cells into an IL-13-producing subset was impaired.

Importantly, these data also demonstrate that PCS abrogates HDM-induced and LPS induced production of CCL20, and reduces TLR4 signalling via LPS.

1. A method of treating and/or preventing an eosinophilic disease or disorder in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

2. The method according to claim 1 wherein the eosinophilic disease or disorder in a subject is selected from the group consisting of a hypereosinophilic syndrome, eosinophilic gastritis, eosinophilic gastroenteritis, eosinophilic esophagitis, eosinophilic pneumonia, eosinophilic granulomatosis with polyangiitis, allergy, dermatitis, asthma and chronic rhinosinusitis.

3. The method according to claim 1 or claim 2 wherein the eosinophilic disease or disorder in a subject is a pulmonary disease or disorder.

4. The method according to claim 1 wherein the eosinophilic disease or disorder in a subject is asthma.

5. The method according to claim 1 wherein the eosinophilic disease or disorder in a subject is allergic airway disease.

6. The method according to claim 1 wherein the eosinophilic disease or disorder in a subject is house dust mite associated allergic airway disease.

7. The method according to any one of claims 1 to 6 wherein the administration of the therapeutically effective amount of the one or more eosinophil antagonist results in reduced eosinophilia.

8. The method according to any one of claims 1 to 6 wherein the administration of the therapeutically effective amount of the one or more eosinophil antagonist results in reduced eosinophilia in bronchoalveolar lavage fluid.

9. The method according to any one of claims 1 to 6 wherein the administration of the therapeutically effective amount of the one or more eosinophil antagonist results in reduced infiltration of pulmonary dendritic cells into the lungs.

10. The method according to any one of claims 1 to 6 wherein the administration of the therapeutically effective amount of the one or more eosinophil antagonist results in reduced activation of pulmonary dendritic cells.

11. The method according to any one of claims 1 to 6 wherein the administration of the therapeutically effective amount of the one or more eosinophil antagonist results in reduced migration of pulmonary dendritic cells into draining lymph nodes of the subject.

12. The method according to any one of claims 1 to 6 wherein the administration of the therapeutically effective amount of the one or more eosinophil antagonist results in reduced goblet cell hyperplasia.

13. The method according to any one of claims 1 to 6 wherein the administration of the therapeutically effective amount of the one or more eosinophil antagonist results in reduced mucus production.

14. The method according to any one of claims 1 to 6 wherein the administration of the therapeutically effective amount of the one or more eosinophil antagonist results in reduced peribronchial and/or perivascular inflammatory cell infiltrate.

15. The method according to any one of claims 1 to 6 wherein the administration of the therapeutically effective amount of the one or more eosinophil antagonist results in reduced infiltration of neutrophils into the lungs.

16. The method according to any one of claims 1 to 6 wherein the administration of the therapeutically effective amount of the one or more eosinophil antagonist results in reduced pathologic change in the lungs.

17. The method according to any one of claims 1 to 6 wherein the administration of the therapeutically effective amount of the one or more eosinophil antagonist results in reduced production of Th2-associated cytokines.

18. The method according to claim 17 wherein the Th2-associated cytokines are IL-5 and/or IL-13

19. The method according to any one of claims 1 to 6 wherein the administration of the therapeutically effective amount of the one or more eosinophil antagonist results in reduced production of allergen-specific antibodies

20. The method according to any one of claims 1 to 6 wherein the administration of the therapeutically effective amount of the one or more eosinophil antagonist results in reduced production of allergen-specific IgE.

21. The method according to any one of claims 1 to 6 wherein the administration of the therapeutically effective amount of the one or more eosinophil antagonist results in reduced production of house dust mite specific antibodies.

22. The method according to any one of claims 1 to 6 wherein the administration of the therapeutically effective amount of the one or more eosinophil antagonist results in reduced production of house dust mite specific IgE.

23. The method according to any one of claims 1 to 6 wherein the administration of the therapeutically effective amount of the one or more eosinophil antagonist results in reduced T cell priming by pulmonary dendritic cells.

24. The method according to any one of claims 1 to 6 wherein the administration of the therapeutically effective amount of the one or more eosinophil antagonist results in reduced CCL20 expression in airway epithelia in the subject.

25. The method according to any one of claims 1 to 6 wherein the administration of the therapeutically effective amount of the one or more eosinophil antagonist results in reduced CCR6 signalling in the subject.

26. The method according to any one of claims 1 to 6 wherein the administration of the therapeutically effective amount of the one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate results in reduced TLR4 signalling in the subject.

27. A method of reducing eosinophilia in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

28. A method of reducing infiltration of pulmonary dendritic cells into the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

29. A method of reducing activation of pulmonary dendritic cells in the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

30. A method of reducing migration of pulmonary dendritic cells into lymph nodes of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

31. A method of reducing goblet cell hyperplasia in the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

32. A method of reducing mucus production in the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

33. A method of a peribronchial and/or perivascular inflammatory cell infiltrate in the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

34. A method of reducing infiltration of neutrophils into the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

35. A method of reducing pathologic change in the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

36. A method of reducing Th2-associated cytokine production in the lungs of a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

37. The method according to claim 36 wherein the Th2-associated cytokine is IL-5 and/or IL-13

38. A method of reducing the production of allergen specific antibodies in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

39. A method of reducing the production of house dust mite specific antibodies in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

40. The method according to claim 38 or claim 39 wherein the antibodies are IgE antibodies.

41. A method of reducing the priming of T cells by pulmonary dendritic cells in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

42. A method of reducing CCL20 expression in airway epithelia in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

43. A method of reducing CCR6 signalling in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

44. A method of reducing TLR4 signalling in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

45. A method according to claim 43 or claim 44 wherein EGFR-TLR4 cross talk is reduced.

46. A method of reducing EGFR mediated signalling in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

47. A method of reducing LPS-induced septic shock in a subject, said method comprising administering to the subject a therapeutically effective amount of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

48. The method according to any one of claims 1 to 47, wherein L-tyrosine, N-acetyl-L-tyrosine, L-DOPA, 4-hydroxyphenylpyruvate, 4-hydroxyphenylacrylate, 3-(p-hydroxyphenyl)propionate, p-cresol and/or p-cresol sulphate is produced in the subject following administration of the one or more eosinophil antagonist.

49. The method according to any one of claims 1 to 48, wherein the therapeutically effective amount of the one or more eosinophil antagonist is administered in two or more doses.

50. The method according to any one of claims 1 to 49, wherein the therapeutically effective amount of the one or more eosinophil antagonist is administered daily, weekly, biweekly, bimonthly, and or quarterly.

51. The method according to any one of claims 1 to 50, wherein the subject is administered with a therapeutically effective amount of the one or more eosinophil antagonist is treated before, during, after, or simultaneously with one or more additional therapies for the treatment of the eosinophilic disease or disorder.

52. The method according to method according to any one of claims 1 to 51, wherein the therapeutically effective amount of the one or more eosinophil antagonist is administered orally, by inhalation, intravenously, intramuscularly, subcutaneously, topically or a combination thereof.

53. The method according to any one of claims 1 to 53, wherein the one or more eosinophil antagonist is formulated as a composition further comprising one or more pharmaceutically acceptable excipients.

54. A composition comprising one or more eosinophil antagonists for use in the treatment and/or prevention of a pulmonary disease in a subject, wherein the one or more eosinophil antagonist is selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

55. The method according to any one of claims 1 to 53, or a composition according to claim 54, wherein the composition consists of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionate.

56. A use of one or more eosinophil antagonist selected from the group consisting of L-phenylalanine, L-tyrosine, N-acetyl-L-tyrosine, L-DOPA, 4-hydroxyphenylpyruvate, 4-hydroxyphenylacrylate, and 3-(p-hydroxyphenyl)propionatein the manufacture of a medicament for treating an eosinophilic disease or disorder in a subject.
